SEQUENCE LISTING (43 sequences)

SEQ ID NO: 1 - DNA, 24 bp, artificial sequence
  /note=“Description of artificial sequencePrimerP1”
cttaccccacatagacctctgaca

SEQ ID NO: 2 - DNA, 22 bp, artificial sequence
  /note=“Description of artificial sequencePrimerP2”
tgctgttggatggtcttcacag

SEQ ID NO: 3 - DNA, 21 bp, artificial sequence
  /note=“Description of artificial sequencesiRNA target sequenceG alpha 13”
cagcaacgtgatcaaaggtat

SEQ ID NO: 4 - DNA, 21 bp, artificial sequence
  /note=“Description of artificial sequencesiRNA target sequenceG alpha 12”
ccgcgacaccatcttcgacaa

SEQ ID NO: 5 - DNA, 21 bp, artificial sequence
  /note=“Description of artificial sequencesiRNA target sequenceG alpha q”
aagcactctttagaaccatta

SEQ ID NO: 6 - DNA, 21 bp, artificial sequence
  /note=“Description of artificial sequencesiRNA target sequenceG alpha 11”
cacaactggcatcatcgagta

SEQ ID NO: 7 - DNA, 19 bp, artificial sequence
  /note=“Description of artificial sequencesiRNA target sequenceArhgef12”
gtctcaagttgtctgagta

SEQ ID NO: 8 - DNA, 19 bp, artificial sequence
  /note=“Description of artificial sequencesiRNA target sequence Mkl1 (MRTF-A)”
caatttgcctccacttagt

SEQ ID NO: 9 - DNA, 19 bp, artificial sequence
  /note=“Description of artificial sequencesiRNA target sequence Mkl2 (MRTF-B)”
cttagaacctgtgaacagt

SEQ ID NO: 10 - DNA, 23 bp, artificial sequence
  /note=“Description of artificial sequencePrimerrat G alpha 12 Forward”
cggcaagtccaccttcctcaagc

SEQ ID NO: 11 - DNA, 21 bp, artificial sequence
  /note=“Description of artificial sequencePrimerrat G alpha 12 Reverse”
tggtgtcgcggaactccagca

SEQ ID NO: 12 - DNA, 20 bp, artificial sequence
  /note=“Description of artificial sequencePrimerrat beta-MHC Forward”
gagggcggacattgccgagt

SEQ ID NO: 13 - DNA, 23 bp, artificial sequence
  /note=“Description of artificial sequencePrimerrat beta-MHC Reverse”
aaggctccaggtctcagggcttc

SEQ ID NO: 14 - DNA, 20 bp, artificial sequence
  /note=“Description of artificial sequencePrimerrat GAPDH Forward”
ggcggcttcggggcacattt

SEQ ID NO: 15 - DNA, 21 bp, artificial sequence
  /note=“Description of artificial sequencePrimer rat GAPDH Reverse”
gggccaggcagttggtggtac

SEQ ID NO: 16 - DNA, 23 bp, artificial sequence
  /note=“Description of artificial sequencePrimermouse ANP Forward”
cacagatctgatggatttcaaga

SEQ ID NO: 17 - DNA, 20 bp, artificial sequence
  /note=“Description of artificial sequencePrimermouse ANP Reverse”
cctcatcttctaccggcatc

SEQ ID NO: 18 - DNA, 21 bp, artificial sequence
  /note=“Description of artificial sequencePrimermouse BNP Forward”
gtcagtcgtttgggctgtaac

SEQ ID NO: 19 - DNA, 20 bp, artificial sequence
  /note=“Description of artificial sequencePrimermouse BNP Reverse”
agacccaggcagagtcagaa

SEQ ID NO: 20 - DNA, 18 bp, artificial sequence
  /note=“Description of artificial sequencePrimermouse beta-MHC Forward”
cgcatcaaggagctcacc

SEQ ID NO: 21 - DNA, 18 bp, artificial sequence
  /note=“Description of artificial sequencePrimermouse beta-MHC Reverse”
ctgcagccgcagtaggtt

SEQ ID NO: 22 - DNA, 20 bp, artificial sequence
  /note=“Description of artificial sequencePrimermouse TGF beta 1 Forward”
tggagcaacatgtggaactc

SEQ ID NO: 23 - DNA, 24 bp, artificial sequence
  /note=“Description of artificial sequencePrimermouse TGF beta 1 Reverse”
cagcagccggttaccaagaccaag

SEQ ID NO: 24 - DNA, 21 bp, artificial sequence
  /note=“Description of artificial sequencePrimermouse Collagen 1 Forward”
catgttcagctttgtggacct

SEQ ID NO: 25 - DNA, 20 bp, artificial sequence
  /note=“Description of artificial sequencePrimermouse Collagen 1 Reverse”
gcagctgacttcagggatgt

SEQ ID NO: 26 - DNA, 20 bp, artificial sequence
  /note=“Description of artificial sequencePrimermouse Collagen 3 Forward”
tcccctggaatctgtgaatc

SEQ ID NO: 27 - DNA, 20 bp, artificial sequence
  /note=“Description of artificial sequencePrimermouse Collagen 3 Reverse”
tgagtcgaattggggagaat

SEQ ID NO: 28 - DNA, 21 bp, artificial sequence
  /note=“Description of artificial sequencePrimermouse Collagen 4 Forward”
ttaaaggactccagggaccac

SEQ ID NO: 29 - DNA, 19 bp, artificial sequence
  /note=“Description of artificial sequencePrimermouse Collagen 4 Reverse”
cccactgagcctgtcacac

SEQ ID NO: 30 - DNA, 18 bp, artificial sequence
  /note=“Description of artificial sequencePrimermouse BAX Forward”
gtgagcggctgcttgtct

SEQ ID NO: 31 - DNA, 20 bp, artificial sequence
  /note=“Description of artificial sequencePrimermouse BAX Reverse”
ggtcccgaagtaggagagga

SEQ ID NO: 32 - DNA, 20 bp, artificial sequence
  /note=“Description of artificial sequencePrimermouse GAPDH Forward”
agcttgtcatcaacgggaag

SEQ ID NO: 33 - DNA, 21 bp, artificial sequence
  /note=“Description of artificial sequencePrimermouse GAPDH Reverse”
tttgatgttagtggggtctcg

SEQ ID NO: 34 - protein, 573 aa, Homo sapiens
MSGTQSTITDRFPLKKPIRHGSILNRESPTDKKQKVERIASHDFDPTGLVQRCVIIQKDDNGFGLTVSGDNPVFVQSVKEDGAAMRAGVQTGDRIIKVNGTLVTHSNHLEVVKLIKSGSYVALTVQGRPPGSPQIPLADSEVEPSVIGHMSPIMTSPHSPGASGNMERITSPVLMGEENNVVHNQKVEILRKMLQKEQERLQLLQEDYNRTPAQRLLKEIQEAKKHIPQLQEQLSKATGSAQDGAVVTPSRPLGDTLTVSEAETDPGDVLGRTDCSSGDASRPSSDNADSPKSGPKERIYLEENPEKSETIQDTDTQSLVGSPSTRIAPHIIGAEDDDFGTEHEQINGQCSCFQSIELLKSRPAHLAVFLHHVVSQFDPATLLCYLYSDLYKHTNSKETRRIFLEFHQFFLDRSAHLKVSVPDEMSADLEKRRPELIPEDLHRHYIQTMQERVHPEVQRHLEDFRQKRSMGLTLAESELTKLDAERDKDRLTLEKERTCAEQIVAKIEEVLMTAQAVEEDKSSTMQYVILMYMKHLGVKVKEPRNLEHKRGRIGFLPKIKQSMKKKKKKKKKK

SEQ ID NO: 35 - protein, 9829 aa, Homo sapiens
GGGAGTCCCGGGTCCCCTTCCCACTGCGCGCGGATTTCCCTCTCTGAGGAAGTTTATCCTTGTGCCTTCTGGAGGATTTCTCTCTAGCTCGACTCACTCTGGACTGACTCGCTCCCTGGCTTTCTCAGTTCGTTTTGGGAGATAACTTGTTTTGCTCCAAGCCGCATCCGTTGACCCCTTACAGTCGGATGGTCTAGATGACTGAATGGAGTTTTGAGTTGGACTTTTGTGTCCCTGACGGAGTTGGGCCTGATCCCAGAGCACTGGGGGTGGGGAGGAGGTGTTACTGTAAAATGCAAGTTGGATAAAAGGAGGACCTCTCGCCAAGGGCCCCAATGAGTGGCACACAGTCTACTATCACCGACAGGTTTCCCCTCAAAAAACCTATAAGGCATGGAAGTATTTTGAACCGAGAGTCACCAACAGATAAGAAGCAGAAAGTTGAGCGCATTGCATCACATGATTTTGACCCCACAGATAGCTCCTCCAAGAAGACAAAGTCTAGTTCAGAGGAGAGTAGATCCGAGATATATGGTCTTGTTCAGCGTTGCGTAATCATCCAGAAAGATGACAATGGATTTGGGCTGACGGTCAGTGGAGACAATCCAGTCTTCGTACAGTCTGTCAAAGAAGATGGAGCAGCCATGCGGGCTGGAGTACAGACAGGTGATCGAATCATCAAGGTGAATGGAACTCTGGTGACTCATTCAAATCATCTGGAGGTGGTGAAGCTAATCAAATCTGGTTCCTATGTAGCTCTCACTGTTCAGGGACGCCCACCTGGGTCGCCCCAGATTCCACTTGCCGACTCTGAAGTAGAGCCGTCAGTCATTGGACATATGTCTCCCATCATGACATCTCCTCATTCACCTGGAGCATCTGGGAATATGGAGAGAATCACTAGTCCTGTGCTCATGGGGGAGGAAAACAATGTGGTTCATAACCAGAAAGTAGAAATTCTGAGAAAAATGTTACAGAAAGAACAGGAACGGCTACAGTTATTGCAGGAAGATTACAACCGAACACCTGCCCAAAGATTGCTAAAAGAGATCCAAGAGGCCAAGAAACACATTCCTCAGCTGCAAGAGCAGTTATCCAAAGCCACAGGCTCTGCTCAGGATGGAGCTGTAGTTACACCCTCCAGACCTTTAGGGGACACCCTAACAGTCAGTGAGGCAGAAACAGATCCTGGAGATGTACTGGGCAGGACTGACTGTAGCAGTGGAGATGCTTCTCGGCCCAGTAGTGACAATGCAGATAGTCCCAAGAGTGGCCCAAAAGAGAGAATTTATCTAGAGGAAAACCCAGAGAAAAGTGAAACAATTCAGGACACTGACACTCAATCACTTGTCGGAAGTCCCTCAACCCGTATAGCACCTCATATTATTGGAGCAGAAGATGATGATTTTGGTACTGAACATGAACAGATCAATGGACAGTGCAGCTGTTTCCAGAGCATTGAATTACTAAAATCTCGCCCGGCTCATTTGGCTGTTTTCTTACACCATGTAGTTTCACAATTTGACCCTGCGACTTTGCTCTGTTATCTCTATTCAGACCTGTATAAACATACCAATTCCAAAGAAACTCGTCGCATCTTCCTTGAGTTTCATCAGTTCTTTCTAGATCGATCAGCACACCTGAAAGTTTCTGTTCCTGATGAAATGTCTGCAGATCTAGAAAAGAGAAGACCTGAGCTCATTCCTGAGGATCTGCATCGCCACTATATCCAAACTATGCAAGAAAGAGTCCATCCAGAAGTTCAAAGGCACTTAGAAGATTTTCGGCAGAAACGTAGTATGGGACTGACCTTGGCTGAAAGCGAGCTGACTAAACTTGATGCAGAGCGAGACAAGGACCGATTGACTTTGGAGAAGGAGCGGACATGTGCAGAACAGATTGTTGCCAAAATTGAAGAAGTATTGATGACTGCTCAGGCTGTAGAGGAAGATAAGAGCTCCACCATGCAGTATGTTATTCTCATGTATATGAAGCATTTGGGAGTAAAAGTGAAAGAGCCTCGAAATTTGGAGCACAAACGGGGTCGGATTGGATTTCTTCCCAAAATCAAGCAAAGTATGAAGAAAGATAAAGAAGGGGAAGAAAAAGGGAAGCGAAGAGGATTCCCCAGCATCCTGGGACCCCCACGGAGACCAAGCCGTCATGACAACAGTGCAATTGGCAGAGCCATGGAACTACAGAAGGCGCGCCACCCTAAGCACTTATCCACACCCTCATCTGTGAGTCCTGAACCTCAGGACTCTGCCAAGTTGCGCCAGAGTGGGTTAGCAAATGAAGGAACAGACGCTGGATACCTGCCTGCCAATTCCATGTCTTCTGTAGCTTCAGGGGCCTCTTTTTCCCAGGAAGGAGGGAAAGAGAATGATACAGGATCAAAGCAAGTTGGAGAAACATCAGCACCTGGAGACACCTTAGATGGCACACCTCGTACTCTCAATACTGTCTTTGATTTCCCACCACCTCCATTAGACCAAGTGCAGGAGGAGGAATGTGAAGTAGAAAGGGTGACTGAACATGGGACACCAAAGCCCTTTCGAAAGTTTGACAGTGTAGCTTTTGGAGAAAGTCAAAGTGAGGATGAACAATTTGAAAATGACTTAGAGACAGATCCACCCAACTGGCAGCAGCTTGTTAGTCGAGAAGTGTTACTGGGACTAAAACCTTGTGAAATCAAAAGACAGGAAGTGATTAATGAATTGTTCTACACTGAAAGAGCTCATGTTCGAACACTGAAGGTTCTTGATCAAGTGTTCTATCAGCGAGTATCCAGAGAAGGAATTCTGTCACCCTCAGAGCTACGGAAAATTTTTTCAAACTTGGAAGATATTCTTCAACTTCATATTGGATTGAATGAACAAATGAAGGCTGTTCGAAAGAGAAATGAGACCTCTGTTATCGATCAGATTGGGGAAGATTTGCTGACATGGTTCAGCGGACCAGGAGAGGAGAAATTGAAACATGCTGCTGCTACCTTTTGCAGTAACCAACCTTTCGCCCTGGAAATGATCAAATCTCGTCAGAAAAAGGATTCTCGATTTCAGACTTTTGTGCAAGATGCTGAAAGTAATCCACTGTGTCGTCGTCTTCAACTGAAGGATATTATTCCCACTCAAATGCAAAGGCTTACTAAGTACCCACTTCTGTTGGATAATATTGCCAAATACACAGAATGGCCAACAGAAAGGGAGAAGGTGAAGAAAGCTGCAGATCACTGTCGTCAGATCTTAAATTATGTAAATCAGGCTGTCAAGGAGGCAGAAAACAAGCAGCGCCTAGAAGATTATCAGCGTCGCCTTGATACCTCCAGCCTGAAGTTGTCAGAGTACCCAAATGTTGAAGAGCTCAGGAATTTGGATTTAACAAAAAGGAAGATGATTCATGAAGGGCCATTGGTTTGGAAGGTGAATAGAGATAAAACTATTGATTTATACACGTTGCTGCTGGAAGACATTCTTGTATTGTTACAAAAGCAGGATGATAGACTGGTTTTAAGGTGTCATAGTAAGATTCTGGCATCTACAGCTGATAGCAAACACACGTTTAGCCCTGTCATTAAGTTGAGTACAGTGTTGGTTCGACAAGTGGCAACAGATAACAAAGCTTTATTCGTCATTTCCATGTCAGACAATGGCGCTCAGATTTATGAACTGGTGGCACAGACAGTTTCTGAAAAGACTGTCTGGCAGGACCTAATCTGTCGGATGGCTGCATCAGTGAAGGAGCAATCCACAAAGCCAATTCCATTACCACAGTCAACACCTGGCGAAGGAGATAATGATGAAGAAGATCCTTCAAAATTAAAAGAGGAGCAGCATGGCATTTCAGTCACTGGTTTGCAGAGTCCAGACAGAGATTTGGGATTAGAATCTACCTTAATATCGTCAAAACCTCAGTCTCATTCACTGAGTACCTCTGGGAAATCAGAGGTACGTGATCTGTTTGTGGCTGAGAGACAGTTTGCAAAGGAACAACATACAGATGGGACACTAAAGGAAGTTGGAGAAGATTATCAAATCGCAATCCCAGATTCACACCTGCCTGTCTCAGAAGAACGGTGGGCATTGGATGCACTAAGAAATTTGGGTTTGTTGAAGCAGTTGCTGGTGCAACAGCTAGGTTTGACTGAGAAGAGCGTTCAGGAAGACTGGCAACATTTCCCAAGATACAGAACAGCCTCTCAGGGGCCGCAGACAGACAGTGTCATCCAGAACTCTGAAAATATTAAGGCCTATCATTCTGGTGAAGGACATATGCCCTTTAGAACTGGAACTGGTGACATTGCAACTTGTTACAGTCCACGGACTTCAACTGAATCTTTTGCTCCACGGGATTCAGTGGGACTGGCACCCCAGGATAGCCAGGCAAGTAACATTTTAGTAATGGACCACATGATTATGACCCCAGAGATGCCTACCATGGAGCCAGAAGGGGGTCTTGATGACAGTGGAGAGCACTTTTTTGATGCCCGTGAAGCACATAGTGATGAGAATCCATCAGAAGGTGATGGAGCAGTTAACAAGGAAGAGAAGGATGTTAATTTACGCATCTCAGGAAACTATTTGATCCTTGATGGCTATGACCCAGTGCAGGAGAGTTCCACAGATGAGGAGGTTGCTTCCTCACTTACCCTGCAGCCCATGACAGGCATCCCTGCTGTGGAATCCACCCACCAGCAGCAACATTCTCCTCAGAATACTCACTCCGATGGGGCAATTTCACCATTCACCCCCGAATTTCTGGTCCAGCAGCGCTGGGGAGCTATGGAGTATTCCTGTTTTGAGATCCAGAGTCCCTCCTCTTGTGCAGATTCACAGAGCCAGATCATGGAGTACATTCATAAGATAGAGGCTGACCTTGAACACTTAAAGAAGGTGGAGGAAAGTTACACCATTCTTTGCCAAAGGCTGGCTGGATCAGCCCTCACAGACAAGCACTCAGATAAAAGTTAGAGCCGCATGTCCTGGAGGTGACTGCAGGTTGTTGGATTTGGAGTATCGGCCGTGTCTCACCACATCCTGGCTCCAGTGTGGATGCAGAGAGAGTGTGACAGAGGATCTGCCTGTGAACCACCTGGGATTAGTCAAGTCCCAAGGTGCCCAGAGTGGGACTAGTTCTTCACAGTGTGGCAGCTGCACTAATCTGTTTGTGAGGGAATATCCATTCCCTCACTCTACTCTCCTCACTATCGGAAATTCATTTTGATTCAGAATAAAAACCAAATGTATAGAGCTTTGGGTGTAGGATATGAAATTGTACTTAGATTTAAGAAAAAGAGAAAATCAGATGTATTTATTTGACTTCATTCCGTATTTGAAAGCACATTTTAATTTTTATTTGCCTTGTTTTGTTTTAATTGAGTAGTGAGAGTTTTAGACCTTTGTCTTTAGTACACCCAAGGATCAACTGCTCCTGAAGCAAAGAGGTCAGGATGGAGTATGCAGAAGTTGGGTAGAGAGGTTAAGAAGAGGAGGAAGTGAGACGGGGAAGGGAGGAGCACCTTGCTTCACTATACAGTATTCCAAGCTCTCTGCTGTCCAGTAGGCTGCTTCTCTGAGGGTCACCTCAAAGGTATGCTATGCCGTGTGGCTCTTATGTGCCCAGGTGGTGTGGTCAGAGAGTGGATGGGCTTCCTCCCGCCCTGAGGCAAGCACCTCTTCTCTGTAGCTGGGATCAACCACAATTAATAGGAATCCTCAACATACTAAATAGCAGGCACTTGAAAATGGGTGTGTTTTCTTCCATTGTCGTCTTTTCTATTGAGGGTTGGGATTTGGGTGGGAGAGGAAAGCAAATTTTATTTTCTTGACTATAAATTTGTTATTCTTGGTATCATTTCATTTTTATAATTATACATTAGACATTGGCACTTGTGTAAAACTGTCCCTGCAGATTGAGCAGGAAGTAAAAACAAATGGAAATGCTTCGGATAGTGGCAGGGGTGGGGGTTGCTAAGGAAAGGGTGGGATGGGAGTGGGTAAAGAGTTTGGGAAGGTTATCTAACTGAATCACTACTGAGTTGAATCATGGCTATCATTAGCCACAGGTACTGCTGATTATAAGGCCAGTAAAATTTTAGTACCTGGAGGTTTGACTCTAATTTTTGCACTGATGGTGCCAAAGGGCCCTGAGTCAAAAGGATAGCCAAGGGTGGAGTGGAAAAAGATATGGTGAAGGCAGAACTGCTCACACCAGCTCCAGAAGCACGTCCTCTGACTTCACTGCCGCAGCTCTTTCCACACGGGGCCGTGATTGACCCTAAAAATTGCAGACCAGCACATACACTGGACACTGCAGGCAAGGTGTTGGTAACTGCCTGCTCTAGGATGAATAGTAGCGTTAGCAGCACCCTACAGAGGGTAAGTTTCAGAATTGTAATTTGAGAACTCCTTCAATTATATTGACTTTCTTTGGTTTTCCTTGTGCATTAGGTATATATTTTTTGAAGTATTTTTGCCAACTAAAATGAAGTCTGTAAATTCTGTTAATAAACAAGGAGTTCATCCGTTGCTCACATCTTTCATTGGTGCCCTCTGAACTCTGTGGGTTGCTAGGATGTAATTTTAATGCTTCCCTGCAGTCCAAAGATGATTTTTTCACCACAAATGGTAAGGGATGCCCACCTACTTTTATAAACACCACTGCAACTTAACAAGTTTATTTATCTATGTCCAGATTTCTGTTTCTGTCCTAAATTGATCTGTGTTTTTAGGTGGATCAACTTGGATCTTTAGACCTCATCTATAAATTGAAATTATATTTTTAGTCATAAGCCAAGTACAATCTAACTCAGAATGGGATTAAAAATTTTAGAAGCAGAAGCTAATATATAAATGAAGTTTGGGATTTGGAACTTTCTGTATCTCTTAGGAGGAACAAGTAAAAACCAAATGGTTACATTGTGCTGCTAGAAATAATGTCATTCTCAATTAAAAGGGTTACACCTGTAGCCACTTGACACTAACACCATCGAGGGCAATTAATCAGGAGAAAAGTAAATATAAAAACTTTGCTCTTAACGTCACACACTACAGGGTAATTATGTAATTCTCTAGCACTCAATTAAGAAGCTTGTAGGTTAGCAAAATTTCAGTTTCTCTAGTTCAGCCAGTGTCTTGTCCTTAGTAATTATTTGCTTTCCCTGCAGTTCAATTTCTCCTTGGAACTCTAACAGGACTAACTGGAAAAATACTTTGACCATAGCTCTAGTACTTCCAAATAAATTCATATCTAGATTTTCAAACAAAGCCCCAAGATGAAGTCTAATTCTGAAAATATCTCACAATTTTTGAATGTTCTTTTTATCTTACCGACCTAACTTCTCTTTTCCCTCCCAACTTGGCCTCCCACACGTTCCATTTCATTTTGGCCATTATAGTCACATCCGTTTGACTTTGGTTGTGACATCTTCCTTTCAGGATGATGGAATTAGATGCAGTCTGTCTTTGAGTGAGACTGGTCACCATCAGAGGAGTTTTTTCTAAGTGACGTGACTAGAAGTTGAAATCTACTTCCCTGCTAAAGGGCACAGGGGTGGTGTACAAAGAAAGCTACCTTCCCAGAGCAAGCAGGCTGCATTTTAGCTATGGAAGTGACCTGCTGTGACACTGTGCTCTCTTCTGTGGGCTTAATGGTTCCTTTGCTATGAAGTGGCAAATTACATGTAGAGTGTCTCCTTCCTTTTCAGAGAACAGTTAATCAAGGCAAATCAGCAAGCCCCCAAAGTGCTGTAATTTAACATCATGATTACCACCTTCGAAGCTATATATTTTGCATACTTTAAAATCACCTAACTTGGACTGCTTGAATTACATTGGCTTTTAGAACCGAAATTGTAACTATGTATTGTATTTCATGGGAGGTATATTTTATGAGCTTTTTGGCTTTCTTTTTTCCCACAGCAACTGCCAATGAAGGATGAATCCCCTTTTTAAAAAGTTGTTGTTGTTGTTGTTTATTTGATTTTGAGTTAGGAGGGATAATAGAGAAGTCCATTTAAAAATTATTTTTAGAAGCTAAAGAAAGTAATTATGCTTCCTGTGAATTGTCTTTTACTGGCATCTTTGTTTTCCTCTTTGATGTTAGTAAATTTGGTGTAATACGTGGGGCTTCCATATTTCAAAGTGGAAGCTTTCTTCTCTGAAGTCGATATATGGTTTTGAATTACTAGAGCTTTGGTCAGTATTTCCTTCCCTATATGTCACAGAGGGCACCACTGAGAACTGCGTGCATAGGACCTCAAAATACAAAATTAGCAGGGCCTCACAGTCAGCTTCCTCATGGCTAGTTTTTCCCCCTTATATTACAATTTTTGTTTTATAAGTCATTTTTTTCCTGATATTTCCACCACTTTTCAGAGTCATCTACAAAATTTTTCTTTCCTCAAGAAAAGAGTTCCTTTTGCCTTATTCCTTATGCCTTCCCCACTGGTATTGAGGGTTTTGATAATAAATTGGTAGGAAAAAAAAGTACCTCCTAGAAGGAAGCCTTCCCCACCATTTCCAGGTGCCAACTGCTAAGCAGATATATTCCAAAAATGGTAACTGTCATGTGCACACTGTTGGTTATTTTTAATAAGCCTCTTCCTACTAGAACATTTTATTTTCCTTGTTCACCATACAATCATGTACTCTTTAACAGAAATTGCTTTTAAAAAATATCTGGAACTATCTTTAAAAAAACTTTATTAATAATCATGTATTTTTACTGATCACATTTTGAAATGCCTAAAAGACTTTATTGTTCTAATTATCCAGATGTACCTTTGTAAAATAGCTCTTTTATGAATTAGCTGATAAGGCTGTATGTTTCTGGAACAAAATATTGGTCATCTAAAAACTTTCTGTTTTCTGGGGTCTGGGAAAATAGAAAATAAGATTTCAAATATTAAATAAGCTTAAAGGAACCAAGCATGTGACTTTTTATTTGTATTCTGGTTGATTTGTCTTGTCCAAGTTATATTAGAAAAGTTGAGGAGTCGAGGAGCCTGTAATTAATTTTCCTGTCAGTGACTGTCAGACATCTTCCTGCTTATTAGAGCATCCCTAGCAACAAGGCTAAACCTTCATGACAGTGCTGGCAGGTGGGCTTGCCCTAGCATGTGGGGCAGGTTTGTTGTTTTTTTAATAGCTTTATTGAGATATTCACATATCATATAATTCATCCATTTAAAGTGTATAATCAGATGGGTTTTGGTATATTATTGATTTTTAATTGTGGTGAAATATATATATAACAAAATTTGCTATTGTAACTATTTTTAAGTATAAAGTTCAGTGAGGTAGGCCAGCTTTTATGACGCACCTGAAGATAACTTTGGTTTTAATCCTACCCTAAATGAAGCATATGCCTCTATTTTTCCTGTCCCCAGTGCTGGGCTTCTTCACATACCAACATTTCTGTGCTTTTACCAACAGCATTTTAGCTCAGAAGGCTCGCTTACTTTGGGCATTTGCTGTATTTTGGTTTTGAAATACTTGTAATTTGACTTATTCTTGGGATAATATTTTAGTTTTCAGTCATTCAAGGGCACATTTGGCTGACGGAGCTAATAAGCGTAATAAAATGCTTAGTAGTTTATACCATCTTTTAGTTAAACATTTTAAAGATTAAGACTATTCAAATGTACTTTGCGGAAATTAACCTGTTTGTATGCCTGGCTTTGTTTACAAACATATATCTATATCTATATATATAGATACAGATACAGATACATATCTGGTGGAACAAAGAAATACCTGTACCATTCCCACTTGCTCTTTGATAGCCACCTCCTAGGAATGGAGACAATAAAACTCTGTATTTGCATGTG

SEQ ID NO: 36 - DNA, 9772 bp, Homo sapiens
gggagtcccgggtccccttcccactgcgcgcggatttccctctctgaggaagtttatccttgtgccttctggaggatttctctctagctcgactcactctggactgactcgctccctggctttctcagttcgttttgggagataacttgttttgctccaagccgcatccgttgaccccttacagtcggatggtctagatgactgaatggagttttgagttggacttttgtgtccctgacggagttgggcctgatcccagagcactgggggtggggaggaggtgttactgtaaaatgcaagttggataaaaggaggacctctcgccaagggccccaatgagtggcacacagtctactatcaccgacaggtttcccctcaaaaaacctataaggcatggaagtattttgaaccgagagtcaccaacagataagaagcagaaagttgagcgcattgcatcacatgattttgaccccacaggtcttgttcagcgttgcgtaatcatccagaaagatgacaatggatttgggctgacggtcagtggagacaatccagtcttcgtacagtctgtcaaagaagatggagcagccatgcgggctggagtacagacaggtgatcgaatcatcaaggtgaatggaactctggtgactcattcaaatcatctggaggtggtgaagctaatcaaatctggttcctatgtagctctcactgttcagggacgcccacctgggtcgccccagattccacttgccgactctgaagtagagccgtcagtcattggacatatgtctcccatcatgacatctcctcattcacctggagcatctgggaatatggagagaatcactagtcctgtgctcatgggggaggaaaacaatgtggttcataaccagaaagtagaaattctgagaaaaatgttacagaaagaacaggaacggctacagttattgcaggaagattacaaccgaacacctgcccaaagattgctaaaagagatccaagaggccaagaaacacattcctcagctgcaagagcagttatccaaagccacaggctctgctcaggatggagctgtagttacaccctccagacctttaggggacaccctaacagtcagtgaggcagaaacagatcctggagatgtactgggcaggactgactgtagcagtggagatgcttctcggcccagtagtgacaatgcagatagtcccaagagtggcccaaaagagagaatttatctagaggaaaacccagagaaaagtgaaacaattcaggacactgacactcaatcacttgtcggaagtccctcaacccgtatagcacctcatattattggagcagaagatgatgattttggtactgaacatgaacagatcaatggacagtgcagctgtttccagagcattgaattactaaaatctcgcccggctcatttggctgttttcttacaccatgtagtttcacaatttgaccctgcgactttgctctgttatctctattcagacctgtataaacataccaattccaaagaaactcgtcgcatcttccttgagtttcatcagttctttctagatcgatcagcacacctgaaagtttctgttcctgatgaaatgtctgcagatctagaaaagagaagacctgagctcattcctgaggatctgcatcgccactatatccaaactatgcaagaaagagtccatccagaagttcaaaggcacttagaagattttcggcagaaacgtagtatgggactgaccttggctgaaagcgagctgactaaacttgatgcagagcgagacaaggaccgattgactttggagaaggagcggacatgtgcagaacagattgttgccaaaattgaagaagtattgatgactgctcaggctgtagaggaagataagagctccaccatgcagtatgttattctcatgtatatgaagcatttgggagtaaaagtgaaagagcctcgaaatttggagcacaaacggggtcggattggatttcttcccaaaatcaagcaaagtatgaagaaagataaagaaggggaagaaaaagggaagcgaagaggattccccagcatcctgggacccccacggagaccaagccgtcatgacaacagtgcaattggcagagccatggaactacagaaggcgcgccaccctaagcacttatccacaccctcatctgtgagtcctgaacctcaggactctgccaagttgcgccagagtgggttagcaaatgaaggaacagacgctggatacctgcctgccaattccatgtcttctgtagcttcaggggcctctttttcccaggaaggagggaaagagaatgatacaggatcaaagcaagttggagaaacatcagcacctggagacaccttagatggcacacctcgtactctcaatactgtctttgatttcccaccacctccattagaccaagtgcaggaggaggaatgtgaagtagaaagggtgactgaacatgggacaccaaagccctttcgaaagtttgacagtgtagcttttggagaaagtcaaagtgaggatgaacaatttgaaaatgacttagagacagatccacccaactggcagcagcttgttagtcgagaagtgttactgggactaaaaccttgtgaaatcaaaagacaggaagtgattaatgaattgttctacactgaaagagctcatgttcgaacactgaaggttcttgatcaagtgttctatcagcgagtatccagagaaggaattctgtcaccctcagagctacggaaaattttttcaaacttggaagatattcttcaacttcatattggattgaatgaacaaatgaaggctgttcgaaagagaaatgagacctctgttatcgatcagattggggaagatttgctgacatggttcagcggaccaggagaggagaaattgaaacatgctgctgctaccttttgcagtaaccaacctttcgccctggaaatgatcaaatctcgtcagaaaaaggattctcgatttcagacttttgtgcaagatgctgaaagtaatccactgtgtcgtcgtcttcaactgaaggatattattcccactcaaatgcaaaggcttactaagtacccacttctgttggataatattgccaaatacacagaatggccaacagaaagggagaaggtgaagaaagctgcagatcactgtcgtcagatcttaaattatgtaaatcaggctgtcaaggaggcagaaaacaagcagcgcctagaagattatcagcgtcgccttgatacctccagcctgaagttgtcagagtacccaaatgttgaagagctcaggaatttggatttaacaaaaaggaagatgattcatgaagggccattggtttggaaggtgaatagagataaaactattgatttatacacgttgctgctggaagacattcttgtattgttacaaaagcaggatgatagactggttttaaggtgtcatagtaagattctggcatctacagctgatagcaaacacacgtttagccctgtcattaagttgagtacagtgttggttcgacaagtggcaacagataacaaagctttattcgtcatttccatgtcagacaatggcgctcagatttatgaactggtggcacagacagtttctgaaaagactgtctggcaggacctaatctgtcggatggctgcatcagtgaaggagcaatccacaaagccaattccattaccacagtcaacacctggcgaaggagataatgatgaagaagatccttcaaaattaaaagaggagcagcatggcatttcagtcactggtttgcagagtccagacagagatttgggattagaatctaccttaatatcgtcaaaacctcagtctcattcactgagtacctctgggaaatcagaggtacgtgatctgtttgtggctgagagacagtttgcaaaggaacaacatacagatgggacactaaaggaagttggagaagattatcaaatcgcaatcccagattcacacctgcctgtctcagaagaacggtgggcattggatgcactaagaaatttgggtttgttgaagcagttgctggtgcaacagctaggtttgactgagaagagcgttcaggaagactggcaacatttcccaagatacagaacagcctctcaggggccgcagacagacagtgtcatccagaactctgaaaatattaaggcctatcattctggtgaaggacatatgccctttagaactggaactggtgacattgcaacttgttacagtccacggacttcaactgaatcttttgctccacgggattcagtgggactggcaccccaggatagccaggcaagtaacattttagtaatggaccacatgattatgaccccagagatgcctaccatggagccagaagggggtcttgatgacagtggagagcacttttttgatgcccgtgaagcacatagtgatgagaatccatcagaaggtgatggagcagttaacaaggaagagaaggatgttaatttacgcatctcaggaaactatttgatccttgatggctatgacccagtgcaggagagttccacagatgaggaggttgcttcctcacttaccctgcagcccatgacaggcatccctgctgtggaatccacccaccagcagcaacattctcctcagaatactcactccgatggggcaatttcaccattcacccccgaatttctggtccagcagcgctggggagctatggagtattcctgttttgagatccagagtccctcctcttgtgcagattcacagagccagatcatggagtacattcataagatagaggctgaccttgaacacttaaagaaggtggaggaaagttacaccattctttgccaaaggctggctggatcagccctcacagacaagcactcagataaaagttagagccgcatgtcctggaggtgactgcaggttgttggatttggagtatcggccgtgtctcaccacatcctggctccagtgtggatgcagagagagtgtgacagaggatctgcctgtgaaccacctgggattagtcaagtcccaaggtgcccagagtgggactagttcttcacagtgtggcagctgcactaatctgtttgtgagggaatatccattccctcactctactctcctcactatcggaaattcattttgattcagaataaaaaccaaatgtatagagctttgggtgtaggatatgaaattgtacttagatttaagaaaaagagaaaatcagatgtatttatttgacttcattccgtatttgaaagcacattttaatttttatttgccttgttttgttttaattgagtagtgagagttttagacctttgtctttagtacacccaaggatcaactgctcctgaagcaaagaggtcaggatggagtatgcagaagttgggtagagaggttaagaagaggaggaagtgagacggggaagggaggagcaccttgcttcactatacagtattccaagctctctgctgtccagtaggctgcttctctgagggtcacctcaaaggtatgctatgccgtgtggctcttatgtgcccaggtggtgtggtcagagagtggatgggcttcctcccgccctgaggcaagcacctcttctctgtagctgggatcaaccacaattaataggaatcctcaacatactaaatagcaggcacttgaaaatgggtgtgttttcttccattgtcgtcttttctattgagggttgggatttgggtgggagaggaaagcaaattttattttcttgactataaatttgttattcttggtatcatttcatttttataattatacattagacattggcacttgtgtaaaactgtccctgcagattgagcaggaagtaaaaacaaatggaaatgcttcggatagtggcaggggtgggggttgctaaggaaagggtgggatgggagtgggtaaagagtttgggaaggttatctaactgaatcactactgagttgaatcatggctatcattagccacaggtactgctgattataaggccagtaaaattttagtacctggaggtttgactctaatttttgcactgatggtgccaaagggccctgagtcaaaaggatagccaagggtggagtggaaaaagatatggtgaaggcagaactgctcacaccagctccagaagcacgtcctctgacttcactgccgcagctctttccacacggggccgtgattgaccctaaaaattgcagaccagcacatacactggacactgcaggcaaggtgttggtaactgcctgctctaggatgaatagtagcgttagcagcaccctacagagggtaagtttcagaattgtaatttgagaactccttcaattatattgactttctttggttttccttgtgcattaggtatatattttttgaagtatttttgccaactaaaatgaagtctgtaaattctgttaataaacaaggagttcatccgttgctcacatctttcattggtgccctctgaactctgtgggttgctaggatgtaattttaatgcttccctgcagtccaaagatgattttttcaccacaaatggtaagggatgcccacctacttttataaacaccactgcaacttaacaagtttatttatctatgtccagatttctgtttctgtcctaaattgatctgtgtttttaggtggatcaacttggatctttagacctcatctataaattgaaattatatttttagtcataagccaagtacaatctaactcagaatgggattaaaaattttagaagcagaagctaatatataaatgaagtttgggatttggaactttctgtatctcttaggaggaacaagtaaaaaccaaatggttacattgtgctgctagaaataatgtcattctcaattaaaagggttacacctgtagccacttgacactaacaccatcgagggcaattaatcaggagaaaagtaaatataaaaactttgctcttaacgtcacacactacagggtaattatgtaattctctagcactcaattaagaagcttgtaggttagcaaaatttcagtttctctagttcagccagtgtcttgtccttagtaattatttgctttccctgcagttcaatttctccttggaactctaacaggactaactggaaaaatactttgaccatagctctagtacttccaaataaattcatatctagattttcaaacaaagccccaagatgaagtctaattctgaaaatatctcacaatttttgaatgttctttttatcttaccgacctaacttctcttttccctcccaacttggcctcccacacgttccatttcattttggccattatagtcacatccgtttgactttggttgtgacatcttcctttcaggatgatggaattagatgcagtctgtctttgagtgagactggtcaccatcagaggagttttttctaagtgacgtgactagaagttgaaatctacttccctgctaaagggcacaggggtggtgtacaaagaaagctaccttcccagagcaagcaggctgcattttagctatggaagtgacctgctgtgacactgtgctctcttctgtgggcttaatggttcctttgctatgaagtggcaaattacatgtagagtgtctccttccttttcagagaacagttaatcaaggcaaatcagcaagcccccaaagtgctgtaatttaacatcatgattaccaccttcgaagctatatattttgcatactttaaaatcacctaacttggactgcttgaattacattggcttttagaaccgaaattgtaactatgtattgtatttcatgggaggtatattttatgagctttttggctttcttttttcccacagcaactgccaatgaaggatgaatcccctttttaaaaagttgttgttgttgttgtttatttgattttgagttaggagggataatagagaagtccatttaaaaattatttttagaagctaaagaaagtaattatgcttcctgtgaattgtcttttactggcatctttgttttcctctttgatgttagtaaatttggtgtaatacgtggggcttccatatttcaaagtggaagctttcttctctgaagtcgatatatggttttgaattactagagctttggtcagtatttccttccctatatgtcacagagggcaccactgagaactgcgtgcataggacctcaaaatacaaaattagcagggcctcacagtcagcttcctcatggctagtttttcccccttatattacaatttttgttttataagtcatttttttcctgatatttccaccacttttcagagtcatctacaaaatttttctttcctcaagaaaagagttccttttgccttattccttatgccttccccactggtattgagggttttgataataaattggtaggaaaaaaaagtacctcctagaaggaagccttccccaccatttccaggtgccaactgctaagcagatatattccaaaaatggtaactgtcatgtgcacactgttggttatttttaataagcctcttcctactagaacattttattttccttgttcaccatacaatcatgtactctttaacagaaattgcttttaaaaaatatctggaactatctttaaaaaaactttattaataatcatgtatttttactgatcacattttgaaatgcctaaaagactttattgttctaattatccagatgtacctttgtaaaatagctcttttatgaattagctgataaggctgtatgtttctggaacaaaatattggtcatctaaaaactttctgttttctggggtctgggaaaatagaaaataagatttcaaatattaaataagcttaaaggaaccaagcatgtgactttttatttgtattctggttgatttgtcttgtccaagttatattagaaaagttgaggagtcgaggagcctgtaattaattttcctgtcagtgactgtcagacatcttcctgcttattagagcatccctagcaacaaggctaaaccttcatgacagtgctggcaggtgggcttgccctagcatgtggggcaggtttgttgtttttttaatagctttattgagatattcacatatcatataattcatccatttaaagtgtataatcagatgggttttggtatattattgatttttaattgtggtgaaatatatatataacaaaatttgctattgtaactatttttaagtataaagttcagtgaggtaggccagcttttatgacgcacctgaagataactttggttttaatcctaccctaaatgaagcatatgcctctatttttcctgtccccagtgctgggcttcttcacataccaacatttctgtgcttttaccaacagcattttagctcagaaggctcgcttactttgggcatttgctgtattttggttttgaaatacttgtaatttgacttattcttgggataatattttagttttcagtcattcaagggcacatttggctgacggagctaataagcgtaataaaatgcttagtagtttataccatcttttagttaaacattttaaagattaagactattcaaatgtactttgcggaaattaacctgtttgtatgcctggctttgtttacaaacatatatctatatctatatatatagatacagatacagatacatatctggtggaacaaagaaatacctgtaccattcccacttgctctttgatagccacctcctaggaatggagacaataaaactctgtatttgcatgtg

SEQ ID NO: 37 - DNA, 19 bp, artificial sequence
  /note=“Description of artificial sequencesiRNA target sequenceArhgef12-II”
ccaagtattctatcagcga

SEQ ID NO: 38 - DNA, 21 bp, artificial sequence
  /note=“Description of artificial sequencesiRNA target sequenceG alpha 13-II”
cagtatcttcctgctataaga

SEQ ID NO: 39 - DNA, 19 bp, artificial sequence
  /note=“Description of artificial sequencesiRNA target sequenceG alpha 12-II”
gtgagtcagtgaagtactt

SEQ ID NO: 40 - DNA, 19 bp, artificial sequence
  /note=“Description of artificial sequencesiRNA target sequenceCD44”
ctacttcactggaaggcta

SEQ ID NO: 41 - DNA, 19 bp, artificial sequence
  /note=“Description of artificial sequencesiRNA target sequenceFYN”
gagaatccctgcagttgat

SEQ ID NO: 42 - DNA, 19 bp, artificial sequence
  /note=“Description of artificial sequencesiRNA target sequenceSRC”
cagcttgtggcttactact

SEQ ID NO: 43 - DNA, 19 bp, artificial sequence
  /note=“Description of artificial sequencesiRNA target sequenceRhoA”
cagacactgatgttatact

(19) United States
(12) Patent Application Publication
(10) Pub. No.: US 2015/0031622 A1
(43) Pub. Date: Jan. 29, 2015

(54) RHOGEF12 IS A THERAPEUTIC TARGET FOR THE TREATMENT OF HEART FAILURE

(71) Applicant: MAX-PLANCK-GESELLSCHAFT ZUR FÖRDERUNG DER WISSENSCHAFTEN E.V., München (DE)

(72) Inventors: Mikito Takefuji, Bad Nauheim (DE); Nina Wettschureck, Bad Nauheim (DE); Stefan Offermanns, Bad Nauheim (DE)

(21) Appl. No.: 14/382,094

(22) PCT Filed: Mar. 1, 2013
(86) PCT No.: PCT/EP2013/054221

§ 371 Date: Aug. 29, 2014

(30) Foreign Application Priority Data
Mar. 1, 2012 (EP) 12157680.5

(51) Int. Cl. C12N 15/115; C07K 14/47; G01N 33/50; C12N 15/113

(52) U.S. Cl. CPC C12N 15/115; C12N 15/113; C07K 14/4705; G01N 33/5023; C12N 2310/16; C12N 2310/14; C12N 2310/531; C12N 2310/141; C12N 2310/12; C12N 2310/11; G01N 2500/10; G01N 2500/04
USPC 514/16.4; 514/44 R; 514/44 A; 435/6.11; 435/6.12; 435/7.1

(57) ABSTRACT

The present invention relates to an inhibitor of the Rho guanine nucleotide exchange factor 12 (RhoGEF12) or an inhibitor of an activator of RhoGEF12 for use in the prevention and/or treatment of heart failure associated with cardiac hypertrophy and/or cardiac fibrosis and diseases associated therewith. Further, it relates also a method of treatment and methods for identifying inhibitors of Rho guanine nucleotide exchange factor 12 (RhoGEF12) or inhibitors of an activator of RhoGEF12.

The present invention relates to an inhibitor of the Rho guanine nucleotide exchange factor 12 (RhoGEF12) or an inhibitor of an activator of RhoGEF12 for use in the prevention and/or treatment of heart failure associated with cardiac hypertrophy and/or cardiac fibrosis and diseases associated therewith. Further, it relates also a method of treatment of said diseases and methods for identifying inhibitors of Rho guanine nucleotide exchange factor 12 (RhoGEF12) or inhibitors of an activator of RhoGEF12.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Structural cardiac remodeling, including cardiac hypertrophy and fibrosis, plays a crucial role in the pathogenesis of heart insufficiency and ensuing heart failures. Myocardial hypertrophy is the adaptive response of the heart to pressure or volume overload, for example in valve disease, hypertension, or after myocardial infarction. Under conditions of prolonged overload, the initially compensatory hypertrophic response may become maladaptive, resulting in chronic heart failure (1). The mechanisms underlying cardiac remodeling have been studied intensively, and in addition to direct mechanical stress as the primary stimulus, a variety of humoral factors have been implicated, for example endothelin-1 (ET-1), angiotensin II (AngII), catecholamines, cytokines, and growth factors (2-4). The receptors of these mediators converge on a limited number of intracellular signaling pathways such as mitogen-activated protein kinase signaling, the PI3K/Akt/GSK-3 pathway, or calcium/calmodulin-dependent calcineurin phosphorylation (2-5). In vitro data also suggest a role for the small GTPase RhoA, a well-known regulator of the actin cytoskeleton (6, 7). RhoA can be activated by adhesion molecules, receptor tyrosine kinases, or G-protein-coupled receptors (GPCRs) (8), but whether any of these pathways is required for cardiomyocyte hypertrophy in vivo, is unclear. Based on in vitro studies it was suggested that hypertrophic stimuli such as ET-1 activate RhoA through G_{q/11} (6), a G-protein family known to play a crucial role in cardiomyocyte hypertrophy (5, 9, 10).

The technical problem underlying the present invention was to identify alternative and/or improved means and methods to therapeutically address heart insufficiency associated with cardiac hypertrophy and/or cardiac fibrosis.

The solution to this technical problem is achieved by providing the embodiments characterized in the claims.

Accordingly, the present invention relates in a first embodiment to an inhibitor of the Rho guanine nucleotide exchange factor 12 (RhoGEF12) or an inhibitor of an activator of RhoGEF12 for use in the prevention and/or treatment of heart failure associated with cardiac hypertrophy and/or cardiac fibrosis and diseases associated therewith.

The term “Rho guanine nucleotide exchange factor 12” is abbreviated as RhoGEF12 and well known in the art (also known as LARG, leukemia-associated Rho guanine nucleotide exchange factor). Briefly, the term relates to a protein encoded by the Arhgef12 gene in mice or the ARHGEF12 gene in humans, that has been shown to mediate RhoA activation by the G_{12/13} family (43, 46) RhoGEF12 contains a RGS homology (RH) domain and a Dbl homology/pleckstrin homology (DH/PH) domain. Activated Gα₁₂ and Gα₁₃ bind RhoGEF12 through the RH domain, triggering a conformational rearrangement of RhoGEF12 which causes the binding to RhoA through the DH/PH domain. The DH/PH domain is directly involved in the catalytic activity of GDP-GTP exchange of RhoA. The amino acid sequence derived from the human RhoGEF12 cDNA sequence has been published in Kourlas et al., 2000 (42) The complete human protein (SEQ ID NO: 34) and mRNA (SEQ ID NO:35 and 36) sequence can be retrieved from the database maintained online by the National Center for Biotechnology Information (NCBI), 8600 Rockville Pike, Bethesda Md., 20894 USA, using the accession number AAH63117.1 (protein sequence, released 3 Jan. 2005) and NM_{—}015313 (transcript variant 1, released 24 Dec. 2011) or NM_{—}001198665.1 (transcript variant 2, released 24 Dec. 2011).

The term “inhibitor” in accordance with the present invention refers to an inhibitor that reduces or abolishes the biological function or activity of a particular target protein, i.e. the Rho guanine nucleotide exchange factor 12 (RhoGEF12) or activator of RhoGEF12. An inhibitor may perform any one or more of the following effects in order to reduce or abolish the biological function or activity of the protein to be inhibited: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in the presence of the inhibitor, and (iv) the protein performs its cellular function with lowered efficiency in the presence of the inhibitor.

Compounds falling in class (i) include compounds interfering with the transcriptional machinery and/or its interaction with the promoter of said gene and/or with expression control elements remote from the promoter such as enhancers. Compounds of class (ii) comprise antisense constructs and constructs for performing RNA interference (e.g. siRNA) well known in the art (see, e.g. Zamore (2001) Nat Struct Biol. 8(9), 746; Tuschl (2001) Chembiochem. 2(4), 239). Compounds of class (iii) interfere with molecular function of the protein to be inhibited, such as receptor signalling activity and activation of downstream target molecules. Accordingly, active site binding compounds are envisaged. Class (iv) includes compounds which do not necessarily bind directly to the target, but still interfere with its function or activity, for example by binding to and/or inhibiting the function or inhibiting expression of members of a pathway which comprises the target. These members may be either upstream or downstream of the target within said pathway. For example, such compounds may alter the affinity or rate of binding of a known ligand to the receptor or compete with a ligand for binding to the receptor or displace a ligand bound to the receptor.

The inhibitor, in accordance with the present invention, may in certain embodiments be provided as a proteinaceous compound or as a nucleic acid molecule encoding the inhibitor. For example, the nucleic acid molecule encoding the inhibitor may be incorporated into an expression vector comprising regulatory elements, such as for example specific promoters, and thus can be delivered into a cell. Methods for targeted transfection of cells and suitable vectors are known in the art, see for example Sambrook and Russel (“Molecular Cloning, A Laboratory Manual”, Cold Spring Harbor Laboratory, N.Y. (2001)). Incorporation of the nucleic acid molecule encoding the inhibitor into an expression vector allows to either selectively or permanently elevate the level of the encoded inhibitor in any cell or a subset of selected cells of a recipient. Thus, a tissue- and/or time-dependent expression of the inhibitor can be achieved, for example restricted to cells of the myocardium. In a preferred embodiment, the inhibitor is therefore a myocardial-specific inhibitor, more preferably a cardiomyocyte-specific inhibitor.

The term “inhibitor of Rho guanine nucleotide exchange factor 12 (RhoGEF12)” in accordance with the present invention refers to an inhibitor that reduces the biological function or activity of RhoGEF12. Biological function or activity denotes in particular any known biological function or activity of RhoGEF12 including those elucidated in accordance with the present invention. Examples of said biological function or activity are the activation of RhoA, its capability of being activated by G-protein alpha subunit Gα₁₃, the induction of the hypertrophic gene program, the induction and maintenance of cardiac hypertrophy as well as cardiac fibrosis resulting in an impaired heart function. All these functions or activities can be tested for either using any of a variety of standard methods known in the art, such as echocardiography, heart catheter and cardiac magnetic resonance tomography, plasma cardiac biomarkers (ANP, BNP), survival rate, electrocardiogram (ECG), or on the basis of the teachings of the examples provided below, optionally in conjunction with molecular techniques such as RT-PCR or with the teachings of the documents cited therein.

In a preferred embodiment, the inhibitor reduces the biological function or activity of RhoGEF12 by at least 50%, preferably by at least 75%, more preferred by at least 90% and even more preferred by at least 95% such as at least 98% or even by 100% as compared to the biological function or activity in the absence of said inhibitor. The term reduction by at least, for example 75%, refers to a decreased biological function or activity such that RhoGEF12 looses 75% of its function or activity and, consequently, has only 25% of the function or activity remaining as compared to an RhoGEF12 protein that is not inhibited. Also preferred, biological function or activity of RhoGEF12 drops to less than 10⁻², less than 10⁻³, less than 10⁻⁴ or less than 10⁻⁵ times the biological function or activity compared to the biological function or activity in the absence of said inhibitor. As outlined herein above, the reduction of the biological function or activity of RhoGEF12 is mediated by inhibitors using different mechanisms of actions. Depending on said mechanism of action, a reduction of, e.g., 75% may be achievable by a given inhibitor by reducing the biological function or activity of all or substantially all RhoGEF12 proteins by 75% or by fully inhibiting 75% of all or substantially all RhoGEF12 proteins. In other words, the reduction of said biological function or activity may be of qualitative or quantitative nature. The term “substantially all” is meant to specify that at least 95% or more of the RhoGEF12 proteins are encompassed. The use of the term “substantially all” is a tribute to the constant changes of protein expression seen in a cell.

The function of any of the inhibitors referred to in the present invention may be identified and/or verified by using, e.g., high throughput screening assays (HTS). High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

The determination of the binding of potential inhibitors can be effected in, for example and without limitation, any binding assay, preferably biophysical binding assay, which may be used to identify binding of test molecules prior to performing the functional/activity assay with the inhibitor. Suitable biophysical binding assays are known in the art and comprise fluorescence polarization (FP) assay, fluorescence resonance energy transfer (FRET) assay and surface plasmon resonance (SPR) assay. Instead of or in addition to assessing the direct interaction of inhibitor and target molecule by binding assays, one may indirectly determine the interaction of the inhibitor with its target molecule by using a suitable read out. For example, in cases where the inhibitor acts by decreasing the expression level of the target protein, the determination of the expression level of the protein can, for example, be carried out on the nucleic acid level or on the amino acid level.

Methods for determining the expression of a protein on the nucleic acid level include, but are not limited to, northern blotting, PCR, RT-PCR or real RT-PCR.

A northern blot allows the determination of RNA or isolated mRNA in a sample. Northern blotting involves the use of electrophoresis to separate RNA samples by size and detection with a hybridization probe complementary to part of or the entire target sequence. Initially, total RNA extraction from the sample is performed. If desired, mRNA can be separated from said initial RNA sample through the use of oligo (dT) cellulose chromatography to isolate only the RNA with a poly(A) tail. RNA samples are then separated by gel electrophoresis. The separated RNA is then transferred to a nylon membrane through a capillary or vacuum blotting system. After transfer to the membrane, the RNA is immobilized through covalent linkage to the membrane by, e.g., UV light or heat. Then, the RNA is detected using suitably labeled probes and X-ray film and can subsequently be quantified by densitometry. Suitable compositions of gels, buffers and labels are well-known in the art and may vary depending on the specific sample and target to be identified.

PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq polymerase. The primers for the amplification may be labeled or be unlabeled. DNA amplification can be performed, e.g., with a Applied Biosystems Veriti® Thermal Cycler (Life Technologies Corporation, Carlsbad, Calif.), C1000™ thermal cycler (Bio-Rad Laboratories, Hercules, Calif.,), or SureCycler 8800 (Agilent Technologies, Santa Clara, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step, e.g. at 55° C. for 1 min as well as a final extension step, e.g. at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, it is well known in the art how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746,121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template.

Both processes can be done without having to open the tube to change or add reagents; only the temperature profile is adjusted between the first cycle (RNA template) and the rest of the amplification cycles (DNA template). The RT reaction can be performed, for example, in a 20 μl reaction mix containing: 4 μl of 5×AMV-RT buffer, 2 μl of oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample. Any of the above mentioned reaction conditions may be scaled up according to the needs of the particular case. The resulting products are loaded onto an agarose gel and band intensities are compared after staining the nucleic acid molecules with an intercalating dye such as ethidium bromide or SybrGreen. A lower band intensity of the sample treated with the inhibitor as compared to a non-treated sample indicates that the inhibitor successfully inhibits the protein.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in real time RT-PCR experiments is a DNA intercalating dye such as SybrGreen for monitoring the de novo synthesis of double stranded DNA molecules.

Methods for the determination of the expression of a protein on the amino acid level include, but are not limited to, western blotting or polyacrylamide gel electrophoresis in conjunction with protein staining techniques such as Coomassie Brilliant blue or silver-staining. The total protein is loaded onto a polyacrylamide gel and electrophoresed. Afterwards, the separated proteins are transferred onto a membrane, e.g. a polyvinyldifluoride (PVDF) membrane, by applying an electrical current. The proteins on the membrane are exposed to an antibody specifically recognizing the protein of interest. After washing, a second antibody specifically recognizing the first antibody and carrying a readout system such as a fluorescent dye is applied. The amount of the protein of interest is determined by comparing the fluorescence intensity of the protein derived from the sample treated with the inhibitor and the protein derived from a non-treated sample. A lower fluorescence intensity of the protein derived from the sample treated with the inhibitor indicates a successful inhibitor of the protein. Also of use in protein quantification is the Agilent Bioanalyzer technique (e.g., Agilent 2100 Bioanalyzer; Agilent Technologies, Santa Clara, Calif.).

The term “inhibitor of an activator of RhoGEF12” as used in accordance with the present invention refers to any inhibitor that does not directly interact with RhoGEF12 but with molecules that directly or indirectly activate one or more of the biological functions or activities of RhoGEF12 and preferably one or more of those functions or activities referred to above or elsewhere in this specification. The inhibition values referred to above for the inhibitor of RhoGEF12 apply mutatis mutandis to the inhibitor of an activator of RhoGEF12.

For example, the inhibitor of an activator of RhoGEF12 may be any compound that reduces the amount of the G-protein α-subunit Gα₁₃ available for binding to and activating RhoGEF12. Such a compound may act on Gα₁₃ directly, such as for example by reducing its expression levels or its binding abilities to RhoGEF12 or it may reduce the level of GTP bound by the G-protein carrying the G-protein α-subunit Gα₁₃ thereby preventing Gα₁₃ activation. Thus, also encompassed by the term inhibitor of an activator of RhoGEF12 are compounds that reduce the level of GTP in a cell, preferably a cardiomyocyte so as to achieve the treatment of heart failure associated with cardiac hypertrophy and/or cardiac fibrosis and diseases associated therewith.

It has been postulated that integrins can somehow modulate RhoA activity. As intgerins, such as, e.g., integrin β₁, are involved in a variety of important molecular pathways in the human body, using integrins as a pharmaceutical target is not a viable option. As such, integrins such as integrin β₁ are preferably not an activator in accordance with the invention.

It is understood by the person skilled in the art that the biological activity or function of RhoGEF12 which renders it suitable as therapeutic target for the treatment of heart failure associated with cardiac hypertrophy and/or fibrosis and diseases associated therewith can be inhibited by one or more inhibitor(s), as described herein above, by directly acting on RhoGEF12 or indirectly by inhibiting activators of RhoGEF12, which are preferably upstream of RhoGEF12, preferably provided that the different inhibitors do not interfere with one another which ca be tested in accordance with methods known in the art and/or described herein. It is preferred that the inhibitors provide an additive effect and, optionally, a synergistic effect in their inhibitory activity. RhoGEF12 is part of a signaling pathway thus making it possible to target molecules being part of said pathway upstream and/or downstream of RhoGEF12 to achieve inhibition of the biological activity or function attributed to RhoGEF12 described herein and known in the art. RhoGEF12 is activated by a G-protein subunit, i.e. the G-protein α-subunit Gα₁₃, which in turn is activated by various G-protein-coupled receptors including the α₁-adrenoceptor, angiotensin AT1 receptor, bombesin BB2 (GRP) receptor, bradykinin B₂ receptor, calcium-sensing (CaR) receptor, cholecystokinin CCK₁, receptor, CXC chemokine (KSHV-ORF74) receptor, endothelin ET_{A}, receptor, endothelin ET_{B} receptor, formyl peptide fMLP receptor, galanin GAL2 receptor, lysophosphatidic acid receptors LPA_{1,2,3}, lysophosphatidylcholine receptor G2A, muscarinic acetylcholine receptors M₁ and M₃, protease-activated receptors PAR1, PAR3 and/or PAR4, serotonin 5-HT_{2C} and 5-HT4 receptors, smoothened, sphingosine-1-phosphate receptors S1P_{2,3,4,5}, tachykinin NK1 receptor, Thromboxane A₂ receptor, thyroid-stimulating hormone (TSH) receptor, vasopressin V₁ₐ receptor (44). Gα₁₃ comprises together with Gα₁₂ the G12 family of heterotrimeric G-proteins (45). Gα₁₃ signaling has also been implicated in the control of cell motility and regulation of the actin cytoskeleton, thereby regulating critical aspects of physiology and pathophysiology (43, 47). Preferably, an inhibitor in accordance with the invention directly or indirectly, but specifically inhibits the biological activity or function of RhoGEF12 and/or Gα₁₃. The term “specifically” in this context refers to the capability of an inhibitor to not have an effect or an essential effect on other molecules than the target molecules. In other words, a corresponding inhibitor does not display cross-reactivity or essentially does not display cross-reactivity. In this context, the term “essentially” is meant to refer to an insignificant or negligible effect. The insignificance or negligibility can be based on functional or quantitative parameters. For example, only a minimal amount of cross-reactivity occurs with a different non-target molecule and/or cross-reactivity occurs, however, with a non-target molecule that is present in insignificant amounts and/or the binding of the inhibitor to the non-target molecule is of no consequence.

Preferably, the inhibitor of the present invention is comprised in a pharmaceutical composition, preferably further comprising a pharmaceutically acceptable carrier, excipient and/or diluent.

The term “pharmaceutical composition”, as used herein, relates to a composition for administration to a patient, preferably a human patient. The pharmaceutical composition of the invention comprises at least one, such as at least two, e.g. at least three, in further embodiments at least four such as at last five of the above mentioned inhibitors. The invention also envisages mixtures of inhibitors of RhoGEF12 or of inhibitors of an activator of RhoGEF12. In cases where more than one inhibitor is comprised in the pharmaceutical composition it is understood that none of these inhibitors has any or any essentially inhibitory effect on the other inhibitors also comprised in the composition. The term “essentially” in this context refers to an insignificant or negligible inhibitory effect. Again, it is preferred that the inhibitors provide an additive effect and, optionally, a synergistic effect in their inhibitory activity.

The composition may be in solid, liquid or gaseous form and may be, inter alia, in a form of (a) powder(s), (a) tablet(s), (a) solution(s) or (an) aerosol(s).

As mentioned above, it is preferred that said pharmaceutical composition comprises a pharmaceutically acceptable carrier, excipient and/or diluent. Examples of suitable pharmaceutical carriers, excipients and/or diluents are well known in the art and include phosphate buffered saline solutions, water, emulsions, such as oil/water emulsions, various types of wetting agents, sterile solutions etc. The carriers, excipients and diluents to some extent depend on the chemical nature of the actual inhibitors and can be chosen by the skilled person according to established protocols. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in the bloodstream such as a coronary artery or directly into the respective tissue. The compositions of the invention may also be administered directly to the target site, e.g., by biolistic delivery to an external or internal target site, preferably the heart. Local administration is preferred over systemic administration to, e.g., minimize the amount of drug used or decrease the risk of adverse side effects, if any. The dosage regimen will be determined by the attending physician and clinical factors. As is well known in the medical arts, dosages for any one patient depend upon many factors, including the patient's size, body surface area, age, the potency and bioavailability of the particular compound to be administered, sex, time and route of administration, general health, and other drugs being administered concurrently. In particular, the potency and mode of action of an inhibitor may dictate not only its dosage, but also its way of administration. For example, inhibitors that due to their mode of action completely abolish the biological activity or function of an RhoGEF12 molecule (or an activator thereof) when binding to the latter, may not be suitable for systemic administration due to the possible occurrence of unwanted side effects, if parameters such as, e.g., bioavailability cannot be sufficiently controlled in the sense of minimizing given unwanted side effects. Pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 0.01 μg to 10 mg units per kilogram of body weight per minute. The continuous infusion regimen may be completed with a loading dose in the dose range of 1 ng and 10 mg/kg body weight.

Progress can be monitored by periodic assessment. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. It is particularly preferred that said pharmaceutical composition comprises further agents known in the art to antagonize heart failure. Since the pharmaceutical preparation of the present invention relies on the above mentioned inhibitors, it is preferred that those mentioned further agents are only used as a supplement, i.e. at a reduced dose as compared to the recommended dose when used as the only drug, so as to e.g. reduce side effects conferred by the further agents. Conventional excipients include binding agents, fillers, lubricants and wetting agents.

The term “heart failure” is well-known in the art to relate to a clinical syndrome in which an abnormality of cardiac heart structure or function is responsible for the inability of the heart to fill with and/or eject blood at a rate commensurate with the requirements of the metabolising tissues (E. Braunwald, Heart failure and cor pulmonale, in Harrison's Principles of Internal Medicine, 16^{th} ed (2005) 1367). Heart failure as used herein is associated with cardiac hypertrophy and/or fibrosis, as an abnormality of the heart structure fully or partially responsible for said inability of the heart to fill with or eject blood at a rate commensurate with the requirements of the metabolising tissues. In other words, the term “associated with” includes heart failures caused by cardiac hypertrophy and/or cardiac fibrosis, and heart failures in which cardiac hypertrophy and/or cardiac fibrosis are not (solely) causative, but are to some extent and besides other factors involved in the onset and/or maintenance/progression of heart failure. It is understood, that only those heart failures associated with cardiac hypertrophy and/or fibrosis that can be treated in accordance with the inhibitor or method of the invention, or the inhibitors identified by the method of the invention are encompassed by the invention. Said heart failure includes abnormalities during systole (systolic heart failure) and/or diastole (diastolic heart failure), abnormalities in the left and/or right ventricle, chronic and/or acute heart insufficiency, low-output heart failure. Heart failure as used herein also includes, but is not limited to, conditions and diseases which per definitionem include cardiac hypertrophy and/or fibrosis, or in addition to which cardiac hypertrophy and/or fibrosis has developed, such as hypertrophic cardiomyopathy; left ventricular noncompaction cardiomyopathy; cardiomyopathies associated with conduction defect and/or ion channel disorders; primary and secondary dilated cardiomyopathy; primary restrictive nonhypertrophied cardiomyopathy, idiopathic cardiomyopathy; tachycardia-induced cardiomyopathy; toxic cardiomyopathy such as caused by alcohol abuse, cocaine use, ephedrine use, chemotherapeutic agent, radiation; restrictive cardiomyopathy secondary to myocardial infiltrative or storage diseases, such as amyloidosis, Gaucher disease, Hurler's disease, Hunter's disease, hemochromatosis, Fabry's disease, glycogen storage disease, Niemann-Pick disease; cardiomyopathy related to endocrine and/or metabolic disorders such as thyroid disorders, hyperparathyroidism, pheochromocytoma, acromegaly, obesity cardiomyopathy; cardiomyopathy related to neuromuscular and/or neurologic disorders such as Friedreich's ataxia, muscular dystrophy, myotonic dystrophy, neurofibromatosis, tuberous sclerosis; cardiomyopathy secondary to nutritional deficiencies such as beriberi, pellagra, kwashiorkor, selenium deficiency, carnitine deficiency as well as cardiomyopathy secondary to autoimmune and/or collagen vascular diseases such as secondary to systemic lupus erythematosus, dermatomyositis, rheumatoid arthritis, scleroderma, polyarteritis nodosa. Further, heart failure associated with cardiac hypertrophy and/or fibrosis may result from a large number of diseases or conditions, including, but not limited to, ischemic heart disease; valvular heart disease; hypertensive cardiomyopathy; sarcoidosis; pulmonary hypertension or obstructive sleep apnea (Maron, et al (2006), Circulation 113:1807; E. Braunwald, E. Heart failure and cor pulmonale, in Harrison's Principles of Internal Medicine, 16^{th} ed (2005) 1367). Preferably, heart failure as described herein above results from or is associated with arterial hypertension, valve disease, aortic coarctation, ischemic heart disease, myocardial infarction, cardiac pressure and volume overload.

Heart failure is one of the most frequent causes of death in western countries and is the common final manifestation of different diseases which becomes morphologically apparent in myocardial remodelling such as the development of hypertrophy. In addition, during the transition to heart failure myocyte degeneration, partially compensated by hypertrophy, has been well documented in patients with dilative cardiomyopathy (DCM) as well as in pressure-overloaded human hearts (Hein et al. 2003; Heling et al. 2000; Schaper et al. 1995; Schaper et al. 1991; Sharma et al. 2004). Mechanical load/stress is considered to be a major mediator of these processes. There is also evidence that secreted morphogens, such as cytokines and growth factors, potently induce in vitro markers of mechanical load/stress such as the fetal gene program, hypertrophic responses as well as reorganization of the contractile and the cytoskeletal apparatus (Ebelt et al. 2007; Eppenberger-Eberhardt et al. 1997; Kubin et al. 1999) in the absence of mechanical load. The term “cardiac hypertrophy” is well known in the art and refers to the increase in the volume of cardiac muscle due to sarcomer replication causing cardiomyocytes to increase in size. Physiologic hypertrophy (athlete's heart) is the normal response to healthy exercise, which results in an increase in the heart's muscle mass and pumping ability. Pathological hypertrophy is the response to stress or disease. Although pathological hypertrophy also leads to an increase in muscle mass to sustain cardiac output in the face of stress, prolonged pathological hypertrophy is associated with a significant increase in the risk for sudden death or progression to heart failure.

The term “fibrosis” in relation to the heart is known in the art to relate to the formation of excess fibrous connective tissue in the heart. Fibrocytes normally secrete collagen and function to provide structural support for the heart. When over-activated this process causes heart fibrosis which leads to increased stiffness of the heart. Cardiac fibrosis is associated with the disruption of normal myocardial structure through excessive deposition of extracellular matrix. Fibrosis in heart is a common feature in patients with advanced cardiac failure.

In accordance with the present invention, it was surprisingly found that the Rho guanine nucleotide exchange factor RhoGEF12 is a factor crucially involved in the regulation of pathological remodeling processes of the heart such as cardiac hypertrophy and/or cardiac fibrosis. Said remodeling processes lead inter alia to changes in the phenotype of cardiomyocytes and thereby negatively functionally affecting the latter that may result in the loss of contractile function and ultimately in heart failure. Previously, no role for RhoGEF12 in cardiac remodelling was described nor could be foreseen on the basis of findings up to the invention. In accordance with the invention, it could be shown in vitro that RhoGEF12 activator Gα₁₃ mediates RhoA activation in response to hypertrophic stimuli. Importantly, this finding was extended in in vivo experiments in that inhibition of Gα₁₃ or its effector RhoGEF12 is shown to i) inhibit the development of cardiac hypertrophy and fibrosis to a significant extent and preserved the ejection fraction as compared to controls; and ii) prevents further deterioration of existing hypertrophy and fibrosis thereby preventing the development of heart failure.

The present invention further relates to a method of preventing and/or treating heart failure associated with cardiac hypertrophy and/or cardiac fibrosis and diseases associated therewith comprising administering a pharmaceutically effective amount of an inhibitor of Rho guanine nucleotide exchange factor 12 (RhoGEF12) or an inhibitor of an activator of RhoGEF12 to a subject in need thereof.

Also with regard to this embodiment, it is preferred that the inhibitor is comprised in a pharmaceutical composition as defined above.

In a preferred embodiment of the inhibitor of the invention or the method of the invention, the inhibitor is an antibody or a fragment or derivative thereof, an aptamer, an siRNA, an shRNA, a miRNA, a ribozyme, an antisense nucleic acid molecule, a small molecule or modified versions of these inhibitors.

The term “antibody” as used in accordance with the present invention comprises, for example, polyclonal or monoclonal antibodies. Furthermore, also derivatives or fragments thereof, which still retain the binding specificity, are comprised in the term “antibody”. Antibody fragments or derivatives comprise, inter alia, Fab or Fab′ fragments as well as Fd, F(ab′)₂, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanized (human antibody with the exception of non-human CDRs) antibodies.

Various techniques for the production of antibodies are well known in the art and described, e.g. in Harlow and Lane (1988) and (1999), loc. cit. Thus, the antibodies can be produced by peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique (as originally demonstrated by Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of RhoGEF12 or an activator of RhoGEF12 (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop in the wild protein, the two cysteins lateral chains being able to form a disulfide bridge. Peptide aptamer selection can be made using different systems, but the most widely used is currently the yeast two-hybrid system. Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The term “peptide” as used herein describes a group of molecules consisting of up to 30 amino acids, whereas the term “polypeptide” as used herein describes a group of molecules consisting of more than 30 amino acids. The group of peptides and polypeptides are referred to together with the term “(poly)peptide”. Also encompassed by the term “(poly)peptide” are proteins as well as fragments of proteins of more than 30 amino acids. The term “fragment of protein” in accordance with the present invention refers to a portion of a protein comprising at least the amino acid residues necessary to maintain the biological activity of the protein. Preferably, the amino acid chains are linear. (Poly)peptides may further form multimers consisting of at least two identical or different molecules. The corresponding higher order structures of such multimers are correspondingly termed homo- or heterodimers, homo- or heterotrimers etc. Furthermore, peptidomimetics of such (poly)peptides where amino acid(s) and/or peptide bond(s) have been replaced by functional analogues are also encompassed by the invention. Such functional analogues include all known amino acids other than the 20 gene-encoded amino acids, such as selenocysteine. The term “(poly)peptide” also refers to naturally modified (poly)peptides where the modification is effected e.g. by glycosylation, acetylation, phosphorylation and similar modifications which are well known in the art.

It is also well known that (poly)peptides are not always entirely linear. For instance, (poly)peptides may be branched as a result of ubiquitination, and they may be circular, with or without branching, generally as a result of post-translation events, including natural processing event and events brought about by human manipulation which do not occur naturally. Circular, branched and branched circular (poly)peptides may be synthesized by non-translational natural processes and by synthetic methods. The modifications can be a function of how the (poly)peptide is made. For recombinant (poly)peptides, for example, the modifications will be determined by the host cells posttranslational modification capacity and the modification signals in the amino acid sequence. Accordingly, when glycosylation is desired, a (poly)peptide should be expressed in a glycosylating host, generally an eukaryotic cell, for example Cos 7, HELA or others. The same type of modification may be present in the same or varying degree at several sites in a given (poly)peptide. Also, a given (poly)peptide may contain more than one type of modification.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang.

Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics). The activity and specificity of siRNAs can be altered by various modifications such as, e.g., by inclusion of a blocking group at the 3′ and 5′ ends, wherein the term “blocking group refers to substituents of that can be attached to oligonucleotides or nucleomonomers, either as protecting groups or coupling groups for synthesis (cf. WO 98/13526, EP 2221377 B1), by inclusion of agents that enhance the affinity to the target sequence such as intercalating agents (e.g., acridine, chlorambucil, phenazinium, benzophenanthirdine), attaching a conjugating or complexing agent or encapsulating it to facilitate cellular uptake, or attaching targeting moieties for targeted delivery.

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference. Accordingly, miRNA may be employed as an inhibitor of RhoGEF12 or an inhibitor of an activator of RhoGEF12.

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

A recent development, also useful in accordance with the present invention, is the combination of an aptamer recognizing a small compound with a hammerhead ribozyme. The conformational change induced in the aptamer upon binding the target molecule is supposed to regulate the catalytic function of the ribozyme.

The term “antisense nucleic acid molecule” is known in the art and refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

A “small molecule” as used herein may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays.

The term “modified versions of these inhibitors” in accordance with the present invention refers to versions of the inhibitors that are modified to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (l) derivatisation of hydroxyl groups to ketales, acetales, or (m) N-acetylation to amides, phenylcarbamates, or (n) synthesis of Mannich bases, imines, or (o) transformation of ketones or aldehydes to Schiff's bases, oximes, acetales, ketales, enolesters, oxazolidines, thiazolidines; or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

In another preferred embodiment of the inhibitor of the invention or the method of the invention, the activator of RhoGEF12 is the G-protein alpha subunit Gα₁₃.

The signaling molecule Gα₁₃ and its functional profile, in particular the interaction with RhoGEF12, have been described herein above. It is understood herein that inhibition of Gα₁₃ results in the inhibition of RhoGEF12 so that the claimed treatment effect is achieved. It is further understood by the skilled person that besides Gα₁₃ there exist further possible targets upstream and downstream of RhoGEF12 and Gα₁₃ that can be a pharmaceutical target for the treatment of the disease defined herein. For example, several receptors can be targeted such as the α₁-adrenoceptor, angiotensin AT1 receptor, bombesin BB2 (GRP) receptor, bradykinin B₂ receptor, calcium-sensing (CaR) receptor, cholecystokinin CCK₁, receptor, CXC chemokine (KSHV-ORF74) receptor, endothelin ET_{A}, receptor, endothelin ET_{B} receptor, formyl peptide fMLP receptor, galanin GAL2 receptor, lysophosphatidic acid receptors LPA_{1,2,3}, lysophosphatidylcholine receptor G2A, muscarinic acetylcholine receptors M₁ and M₃, protease-activated receptors PAR1, PAR3 and/or PAR4, serotonin 5-HT_{2c} and 5-HT4 receptors, smoothened, sphingosine-1-phosphate receptors S1P_{2,3,4,5}, tachykinin NK1 receptor, Thromboxane A₂ receptor, thyroid-stimulating hormone (TSH) receptor, vasopressin V₁ₐ receptor (44).

In a further embodiment, the invention relates to a method of identifying an inhibitor of Rho guanine nucleotide exchange factor 12 (RhoGEF12) or an inhibitor of an activator of RhoGEF12 suitable as a lead compound and/or as a medicament for the prevention and/or treatment of heart failure associated with cardiac hypertrophy and/or cardiac fibrosis and diseases associated therewith, comprising the steps of: (a) determining the level of RhoGEF12 protein, the level of RhoGEF12 transcript and/or the level of activity of RhoGEF12 in a cell; (b) contacting said cell or a cell of the same cell population with a test compound; (c) determining the level of RhoGEF12 protein, the level of RhoGEF12 transcript and/or the level of activity of RhoGEF12 in said cell after contacting with the test compound; and (d) comparing the level of RhoGEF12 protein, the level of RhoGEF12 transcript and/or the level of activity of RhoGEF12 determined in step (c) with the RhoGEF12 protein, the RhoGEF12 transcript and/or the RhoGEF12 activity level determined in step (a) and/or with established standard values (for the cell type used), wherein a decrease of the RhoGEF12 protein, the RhoGEF12 transcript and/or the RhoGEF12 activity level in step (c) as compared to step (a) indicates that the test compound is an inhibitor of RhoGEF12 or an inhibitor of an activator of RhoGEF12 suitable as a lead compound and/or as a medicament for the prevention and/or treatment of heart failure associated with cardiac hypertrophy and/or cardiac fibrosis and diseases associated therewith.

Also envisaged is a cell-free method of identifying an inhibitor of Rho guanine nucleotide exchange factor 12 (RhoGEF12) analogously to the above method, if one is to screen for inhibitors which bind to RhoGEF12 and as such act as direct inhibitors. A cell-free fluorophore-based assay for measuring nucleotide exchange on GTPases in either 96-well or 384-well format is commercially available (Cytoskeleton).

This embodiment relates to a cellular screen, wherein inhibitors may be identified which exert their inhibitory activity by interfering with the expression of RhoGEF12, either by affecting the stability (half-life) of RhoGEF12 protein or RhoGEF12 transcript (mRNA) or by interfering with the transcription or translation of RhoGEF12.

The inhibitor can be any of the inhibitors defined above, i.e. an antibody or a fragment or derivative thereof, an aptamer, a siRNA, a shRNA, a miRNA, a ribozyme, an antisense nucleic acid molecule, modified versions of these inhibitors or a small molecule. The inhibitor may further be, for example a (poly)peptide such as a soluble peptide, including Ig-tailed fusion peptides and members of random peptide libraries (see, e.g., Lam et al. (1991) Nature 354: 82-84; Houghten et al. (1991) Nature 354: 84-86) and combinatorial chemistry-derived molecular libraries made of D- and/or L-configuration amino acids or phosphopeptides (e.g., members of random and partially degenerate, directed phosphopeptide libraries, see, e.g., Songyang et al. (1993) Cell 72: 767-778).

The term “said cell or a cell of the same cell population” as used herein refers either to the cell used in step (a) or to a cell being of the same origin as the cell of step (a) and that is identical or essentially identical in its characteristics to the cell of (a). Furthermore, this term also encompasses cell populations, such as for example homogenous or essentially homogenous cell populations consisting of cells having identical or essentially identical characteristics, and, thus, is not restricted to single cell analyses. “Essential identical” means certain differences may exist which are, however, negligible or insignificant with regard to the performance of the cell in the method of the invention. The term “essentially homogenous” is meant to refer to those instances where it is not possible to generate a an entirely pure (homogenous) cell population from a tissue or cell colony due to technical restrictions. In corresponding cell populations the vast majority of cells or homologous, while only a neglibilble or insignificant number of cells are different from said vast majority. The same limitations and definitions with regard to the term “cell” given herein above apply also to this embodiment mutatis mutandis.

“Established standard values” are values that have previously been generated in the respective cells used in the assay in the absence of a test compound, i.e. in untreated cells, and are at the disposal of the person implementing the method of the invention. A corresponding setup may prove beneficial in particular in view of high throughput screenings (HTS).

The cell in this embodiment can be any animal cell. It is understood by the person skilled in the art that depending on the goal to be achieved with the method described, different cells may be more suitable than others. Preferably, the cell is a mammalian cell, more preferred a human cell which is not a human embryonic stem cell. The term “mammalian cell” as used herein, is well known in the art and refers to any cell belonging to an animal that is grouped into the class of mammalia. The term “cell” as used herein can refer to a single and/or isolated cell or to a cell that is part of a multicellular entity such as a tissue, an organism or a cell culture. In other words the method can be performed in vivo, ex vivo or in vitro. Depending on the particular goal to be achieved with the method of the invention, cells of different mammalian subclasses such as prototheria or theria may be used. For example, within the subclass of theria, preferably cells of animals of the infraclass eutheria, more preferably of the order primates, artiodactyla, perissodactyla, rodentia and lagomorpha are used in the method of the invention. Furthermore, within a species one may choose a cell to be used in the method of the invention based on the tissue type and/or capacity to differentiate equally depending on the goal to be achieved by altering the genome. Three basic categories of cells make up the mammalian body: germ cells, somatic cells and stem cells. A germ cell is a cell that gives rise to gametes and thus is continuous through the generations. Stem cells can divide and differentiate into diverse specialized cell types as well as self renew to produce more stem cells. In mammals there are two main types of stem cells: embryonic stem cells and adult stem cells. Somatic cells include all cells that are not a gametes, gametocytes or undifferentiated stem cells. The cells of a mammal can also be grouped by their ability to differentiate. A totipotent (also known as omnipotent) cell is a cell that is able to differentiate into all cell types of an adult organism including placental tissue such as a zygote (fertilized oocyte) and subsequent blastomeres, whereas pluripotent cells, such as embryonic stem cells, cannot contribute to extraembryonic tissue such as the placenta, but have the potential to differentiate into any of the three germ layers endoderm, mesoderm and ectoderm. Multipotent progenitor cells have the potential to give rise to cells from multiple, but limited number of cell lineages. Further, there are oligopotent cells that can develop into only a few cell types and unipotent cells (also sometimes termed a precursor cell) that can develop into only one cell type. There are four basic types of tissues: muscle tissue, nervous tissue, connective tissue and epithelial tissue that a cell to be used in the method of the invention can be derived from, such as for example hematopoietic stem cells or neuronal stem cells. To the extent human cells are envisaged for use in the method of the invention, it is preferred that such human cell is not obtained from a human embryo, in particular not via methods entailing destruction of a human embryo. On the other hand, human embryonic stem cells are at the skilled person's disposal such as taken from existent embryonic stem cell lines commercially available. Accordingly, the present invention may be worked with human embryonic stem cells without any need to use or destroy a human embryo. Alternatively, or instead of human embryonic stem cells, pluripotent cells that resemble embryonic stem cells such induced pluripotent stem (iPS) cells may be used, the generation of which is state of the art (Hargus G et al., Proc Natl Acad Sci USA 107:15921-15926; Jaenisch R. and Young R., 2008, Cell 132:567-582; Saha K, and Jaenisch R., 2009, Cell Stem Cell 5:584-595.

Cells to be used may originate from established cell lines but may also include cells of a primary cell line established from a tissue sample. Preferably, the cells originate from the same cell line or are established from the same tissue. Methods to obtain samples from various tissues and methods to establish primary cell lines are well-known in the art (Jones G E, Wise C J., “Establishment, maintenance, and cloning of human dermal fibroblasts.” Methods Mol Biol. 1997; 75:13-21). Suitable cell lines may also be purchased from a number of suppliers such as, for example, the American tissue culture collection (ATCC), the German Collection of Microorganisms and Cell Cultures (DSMZ) or PromoCell GmbH, Sickingenstr. 63/65, D-69126 Heidelberg.

As outlined above, certain cells may be more suitable than others to determine RhoGEF12 expression levels. For example, the phenotype and physiological state of a specific cell may be more suitable to achieve a pronounced decrease in RhoGEF12 expression. For example, some cells may endogenously not display a very high RhoGEF12 expression level so that the effects of test compounds cannot be decisively determined. Therefore, preferably cells with an RhoGEF12 expression level that is well above the detection threshold of the method for determining expression level so that an accurate determination of the effect of a given test compound can be determined. Alternatively, the RhoGEF12 expression level may be artificially increased in cells endogenously expressing RhoGEF12 at a low level prior to using corresponding cells in the method of the invention. Also, cells may be genetically engineered to express RhoGEF12 or express RhoGEF12 in sufficient amounts.

Preferred cell types that may be used in accordance with the invention are cardiomyocytes, HEK 293 cells or MDCL cells.

As described hereinabove, RhoGEF12 plays a key role in the transition to heart failure associated with cardiac hypertrophy and/or cardiac fibrosis. Therefore, the use of RhoGEF12 or an activator of RhoGEF12 as a target for the discovery of inhibitors suitable for the treatment and/or prevention of heart failure as defined herein is also encompassed by the present invention. It is envisaged that, for example, a decrease of expression levels of RhoGEF12 conferred by an inhibitor as described above may contribute to protection from heart failure as defined herein and may ameliorate diseases/conditions associated therewith, as described above. Accordingly, measurement of the RhoGEF12 protein or RhoGEF12 transcript level may be used as a readout of the above-described assay.

For example, the above-mentioned cell may exhibit a detectable level of RhoGEF12 protein or RhoGEF12 transcript before contacting with the test compound and the level of RhoGEF12 protein or RhoGEF12 transcript may be lower or undetectable after contacting the cell with the test compound, indicating an inhibitor suitable for the treatment and/or prevention of heart failure as defined herein or as a lead compound for the development of a compound for the treatment of heart failure as defined herein. Preferably, the level of RhoGEF12 protein or RhoGEF12 transcript after contacting the cell with the test compound is reduced by, for example, at least 10, at least 20, at least 30, at least 40 or at least 50% as compared to the level of RhoGEF12 protein or RhoGEF12 transcript before contacting the cell with the test compound. More preferably, the level of RhoGEF12 protein or RhoGEF12 transcript after contacting the cell with the test compound is reduced by, for example, at least 60, at least 70, at least 80, at least 90 or at least 95% as compared to the level of RhoGEF12 protein or RhoGEF12 transcript before contacting the cell with the test compound. Most preferably, the level of RhoGEF12 protein or RhoGEF12 transcript after contacting the cell with the test compound is reduced by 100% as compared to the level of RhoGEF12 or RhoGEF12 before contacting the cell with the test compound. The term “the level of RhoGEF12 protein or RhoGEF12 transcript is reduced by (at least) . . . %” refers to a relative decrease compared to the level of RhoGEF12 or RhoGEF12 transcript before contacting the cell with the test compound. For example, a reduction of at least 40% means that after contacting the cell with the test compound the remaining level of RhoGEF12 protein or RhoGEF12 transcript is only 60% or less as compared to the level of RhoGEF12 protein or RhoGEF12 transcript before contacting the cell with the test compound. A reduction by 100% means that no detectable level of RhoGEF12 protein or RhoGEF12 transcript remains after contacting the cell with the test compound.

Measurements of protein levels as well as of transcript level can be accomplished in several ways, as described above.

In a preferred embodiment, the method is carried out in vitro. In vitro methods offer the possibility of establishing high-throughput assays, as described above.

In a yet further embodiment, the invention relates to a method of identifying an inhibitor of Rho guanine nucleotide exchange factor 12 (RhoGEF12) or an inhibitor of an activator of RhoGEF12 suitable as a lead compound and/or as a medicament for the prevention and/or treatment of heart failure associated with cardiac hypertrophy and/or cardiac fibrosis and diseases associated therewith, comprising the steps of: (a) determining the level of activity of an RhoGEF12 target molecule in a cell containing RhoGEF12; (b) contacting said cell or a cell of the same cell population with a test compound; (c) determining the level of activity of the RhoGEF12 target molecule in said cell after contacting with the test compound; and (d) comparing the level of activity of the RhoGEF12 target molecule determined in step (c) with the level of activity of the RhoGEF12 target molecule determined in step (a) and/or with established standard values (for the cell type used), wherein a decreased activity of the target molecule in step (c) as compared to step (a) indicates that the test compound is an inhibitor of RhoGEF12 or an inhibitor of an activator of RhoGEF12 suitable as a lead compound and/or as a medicament for the prevention and/or treatment of heart failure associated with cardiac hypertrophy and/or cardiac fibrosis and diseases associated therewith.

Also envisaged is a cell-free method of identifying an inhibitor of Rho guanine nucleotide exchange factor 12 (RhoGEF12) analogously to the above method, if one is to screen for inhibitors which bind to RhoGEF12 and as such act as direct inhibitors.

This embodiment relates to a cellular screen, wherein inhibitors may be identified which exert their inhibitory activity by physically interacting with RhoGEF12, or alternatively (or additionally) by functionally interacting with RhoGEF12, i.e., by interfering with the pathway(s) present in the cells employed in the cellular assay. Furthermore, such compounds may, as described above, alter the stability, affinity or rate of binding of a known interaction partner, such as Gα₁₃, to RhoGEF12 or compete with a known interaction partner for binding to RhoGEF12 or displace a known interaction partner bound to RhoGEF12. As a result, the biological activity of RhoGEF12 is altered either directly or indirectly, which can be measured as an altered level of activity of an RhoGEF12 target molecule. As used herein, the stability of a molecule may be affected, e.g., by targeting said molecule to proteasomal degradation.

The term “RhoGEF12 target molecule” refers to molecules that are affected by RhoGEF12 activity. For example, the RhoGEF12 target molecule can be a molecule affected by RhoGEF12 activity as a result of the downstream signalling of this molecule. Downstream signalling refers to the modulation (e.g., stimulation or inhibition) of a cellular function/activity upon binding of one interaction partner to another interaction partner, being part of a signalling pathway. Examples of such functions include mobilization or activation of intracellular molecules that participate in a signal transduction pathway, such as for example the small GTPase RhoA. Activation of RhoA has been shown to promote actin polymerization by two downstream signalling modules, one involving the Rho-associated kinase (RoCK)-LiM kinase-cofilin pathway, and the other mediated by formins (48). At low actin polymerization states, myocardin-related transcription factors (MRTFs) are held in an inactive state in the cytoplasm by reversible complex formation with globular actin (G-actin). Polymerization of G-actin into filamentous (F-) actin was suggested to liberate MRTFs from G-actin, thereby allowing the nuclear import of MRTF and subsequent activation of serum response factor (SRF)-dependent gene transcription, for example of atrial natriuretic peptide (ANP) or β-myosin heavy chain (β-MHC) In addition, active RhoA was shown control sarcomere organization and myofibrillogenesis (49; 50).

The “level of activity” of an RhoGEF12 target molecule may be altered due to a change in the biological activity of the RhoGEF12 target molecule. Alternatively, the “level of activity” may also be altered by reducing or increasing the expression level of the RhoGEF12 target molecule. In accordance with the invention, the alteration of the “level of activity” of the RhoGEF12 target molecule is decreased by a test compound to be considered an inhibitor of RhoGEF12 or an inhibitor of an activator of RhoGEF12.

Assays determining the expression of genes that are up- or down-regulated in response to a receptor protein dependent signal cascade can be employed. For example, any of the methods described above for determining the expression of a protein on the protein or the nucleic acid level may be used. Furthermore, the regulatory region of target genes may be operably linked to a marker that is easily detectable, such as for example luciferase, green fluorescent protein (GFP), yellow fluorescent protein (YFP), red fluorescent protein (RFP), cyan fluorescent protein (CFP) or β-galactosidase. Alternatively, for target molecules that have an altered activity upon phosphorylation, the phosphorylation level of said molecules may be measured.

In a preferred embodiment of the method of the invention, the RhoGEF12 target molecule is selected from the GTPase RhoA and the serum response factor (SRF).

All of these molecules are defined in accordance with the present invention in the same manner as known in the prior art and the common general knowledge of the skilled person. Briefly, a transcriptional activator, serum response factor (SRF), has been shown to be involved in the downstream mechanisms by which RhoA activates the hypertrophic gene program. Targeted deletion of SRF in the developing heart reduces expression of many cardiac-specific genes and activation of SRF leads to heart hypertrophy with increased fetal cardiac gene expression. The data generated by the inventors shows that Rhogef12 activates SRF in heart through RhoA signaling. Intracellular levels and activity of these molecules can be determined using methods well known in the art, for example using any of the methods described above. For example, the activity of the GTPase RhoA can be measured using an ELISA kit as outlined herein below in the example section. The RhoA ELISA kit contains a Rho-GTP-binding domain (RBD) of RhoA effector, which binds with active RhoA. The degree of RhoA activation is determined by examining the amount of RhoA in cell lysates bound with RBD. The activity of the serum response factor (SRF) can be determined, e.g., by a luciferase reporter assay system. The luciferase reporter assay system is a technology where a SRF gene is synthesized in response to activation of RhoA, followed by monitoring of the SRF protein expression by its enzymatic activities linked with luminescent read-outs.

In another preferred embodiment of the methods of the invention, said cell comprising RhoGEF12 protein and/or transcript is a cardiomyocyte.

The term “cardiomyocyte” known in the art to refer to cardiac myocytes containing sarcomeric structures. It is understood by the skilled person that it may be advantageous to perform the method for identifying inhibitors according to the invention in cell types playing a role in the disease to be treated. Isolated cardiomyocytes from neonatal rats or mice may be used to study the effect of inhibitors on hypertrophic/fibrotic responses. To this purpose, cultured neonatal cardiomyocytes may either be stimulated with agonists such as Angiotensin II, Endothelin-1, or Phenylephrine, or stretched using, for example, a Flexcell system (Flexcell, Hillsborough, N.C., USA).

Therefore, cardiomyocytes may be used in accordance with the method of the invention, for example, as initial test system or as subsequent test system to evaluate the inhibitory action of a test compound identified in a first screening method according to the invention which did not employ cardiomyocytes.

In a yet other preferred embodiment of the methods of the invention, said methods comprise the further step of optimising the pharmacological properties of an inhibitor of Rho guanine nucleotide exchange factor 12 (RhoGEF12) or an inhibitor of an activator of RhoGEF12 identified as lead compound.

The identified so-called lead compounds may be optimized to arrive at a compound which may be used in a pharmaceutical composition. Methods and tools for the optimization of the pharmacological properties of compounds identified in screens, the lead compounds, are known in the art. For example, in silico tools for optimizing lead compounds are known in the art and described, e.g., in Cruciani et al., European Journal of Pharmaceutical Sciences, vol. 11, suppl. 2, p. S29-S39 (2000). Furthermore, high-throughput approaches for evaluating properties of lead compounds have been described in Tarbit and Berman, Current Opinion in Chemical Biology, vol. 2, issue 3, p. 411-416 (1998).

In a more preferred embodiment of the methods of the invention, the optimisation comprises modifying the inhibitor of RhoGEF12 or the inhibitor of an activator of RhoGEF12 to achieve: i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (l) conversion of alkyl substituents to cyclic analogues, or (m) derivatisation of hydroxyl groups to ketales, acetales, or (n) N-acetylation to amides, phenylcarbamates, or (o) synthesis of Mannich bases, imines, or (p) transformation of ketones or aldehydes to Schiff's bases, oximes, acetales, ketales, enolesters, oxazolidines, thiazolidines or combinations thereof.

The various steps recited above are generally known in the art, as described above.

In a preferred embodiment of the inhibitor or the methods of the invention, the heart insufficiency associated with myocardial hypertrophy and/or myocardial fibrosis results from diseases and conditions selected from the group consisting of arterial hypertension, valve disease, aortic coarctation, cardiomyopathy and myocardial infarction.

Hypertension is a chronic medical condition in which the blood pressure is elevated. Hypertension is classified as either primary hypertension or secondary hypertension; about 95% of cases are categorized as primary hypertension. Hypertension affects about 35% of the US population. Many patients with hypertension have no symptoms, but hypertension is a major risk factor for heart hypertrophy and heart failure.

Valve disease is any disease process involving one or more of the valves of the heart (the aortic and mitral valves on the left and the pulmonary and tricuspid valves on the right). The common types of valve disease are valve stenosis and valve insufficiency. Valve stenosis causes pressure-overload heart failure and valve insufficiency causes volume-overload heart failure. A basic treatment for valve disease is valve replacement. There are two basic types of artificial heart valve: mechanical valves and tissue valves. Tissue valves are usually made from porcine or tissue valves. The tissue valve recipient does not need to take anticoagulant, but the valves typically last 10-15 years. When a tissue valve wears out, the patient must undergo another valve replacement surgery. Although mechanical valves are long-lasting, there is an increased risk of blood clots with mechanical valves. Mechanical valve recipients must take anticoagulant drugs for the rest of their lives. When the mechanical valve recipient has another disease such a cerebral hemorrhage and undergo another surgery, anticoagulant drug increase the risk of bleeding. The valve replacement can recover the recipient from the first stage of heart failure, but the replacement does not redress the patient with severe heart failure. The medication is an important therapy to keep heart function of the patient and postpone the surgery as much as possible.

Aortic coarctation is a narrowing of the aorta in the area of the ductus arteriosus, typically after the left subclavian artery. Aortic coarctation is usually due to a congenital defect. Clinical symptoms depend on location and severity of the coarctation, they often include arterial hypertension in the arteries of head and arms, with normal or low blood pressure in the lower extremities.

Myocardial infarction results from the interruption of blood supply to a part of heart. Percutaneous coronary intervention (PCI) and/or fibrinolysis are recommended within a few hours after heart attack as basic treatment for acute phase of myocardial infarction. After the patient escape from acute phase of myocardial infarction, the series of histopathological and structural changes occur in the ventricular myocardium that lead to chronic heart failure. The medication for ventricular remodeling improves a long-term prognosis.

In another preferred embodiment of the inhibitor or the methods of the invention, the diseases associated with said heart insufficiency are selected from the group consisting of pressure or volume overload-induced heart failure, dyspnea, exercise intolerance, pulmonary edema, peripheral edema, ascites and hepatomegaly.

Also, the invention relates to a method of preventing or reducing hypertrophy and/or fibrosis of cardiomyocytes comprising: contacting said cardiomyocytes with an effective amount of an inhibitor of the Rho guanine nucleotide exchange factor 12 (RhoGEF12) or an inhibitor of an activator of RhoGEF12.

All preferred embodiments, definitions and combinations of technical features mentioned herein above apply mutatis mutandis to this embodiment. The method can be performed in vivo, in vitro, or ex vivo.

In another embodiment, the invention relates to a tamoxifen-inducible cardiomyocyte-specific transgenic RhoGEF12-knockout, or Gα₁₃-knockout or Gα_{12/13}-double knockout mouse.

Mice with cardiomyocyte-specific deficiency for RhoGEF12 (also referred to herein as cmc-Gef12-KO) Gα₁₃ (also referred to herein as cmc-Gα₁₃-KO) or Gα_{12/13} can, e.g., be generated by using a Cre-transgenic mouse line in which a fusion protein of the codon-improved recombinase Cre (iCre) and a tamoxifen-inducible estrogen receptor mutant (CreERT2) is expressed under control of the cardiomyocyte-specific α-myosin heavy chain (αMHC) promoter present on a bacterial artificial chromosome (BAC). β-galactosidase staining of tissue sections from αMHC-CreERT2/Rosa26LacZ double transgenic mice can be used to show Cre-mediated recombination in the heart after tamoxifen treatment, whereas no recombination is observed in other organs.

αMHC-CreERT2 mice can the be crossed to animals carrying a floxed version of Gna13,³⁴ the gene coding for Gα₁₃, or the gene coding for RhoGEF12, respectively, or in some cases also to constitutively Gα₁₂-deficient mice⁵⁵ to produce cardiomyocyte-specific RhoGEF12, Gα₁₃- and Gα₁₃-double-deficient animals (cmc-Gα₁₃-KO and cmc-Gα_{12/13}-DKO, respectively). To induce Cre-mediated recombination of floxed Gna13 or RhoGEF12 alleles, animals can be treated at an age of 6-8 weeks with intraperitoneal injections of 1 mg tamoxifen on 5 consecutive days, and the efficiency of cardiomyocyte-specific recombination can be determined two weeks later by Western blot analysis.

It is understood that the tamoxifen-inducible cardiomyocyte-specific transgenic RhoGEF12, Gα₁₃-knockout or Gα_{12/13}-double knockout mouse can relate to said mouse before tamoxifen administration, i.e. with the genes coding for RhoGEF12, Gα₁₃ or Gα_{12/13} in cardiomyocytes, or after tamoxifen administration, i.e. without the genes coding for RhoGEF12, Gα₁₃ or Gα_{12/13} in cardiomyocytes.

All preferred embodiments, definitions and combinations of technical features mentioned herein above apply mutatis mutandis to this embodiment. The method can be performed in vivo, in vitro, or ex vivo.

The figures show:

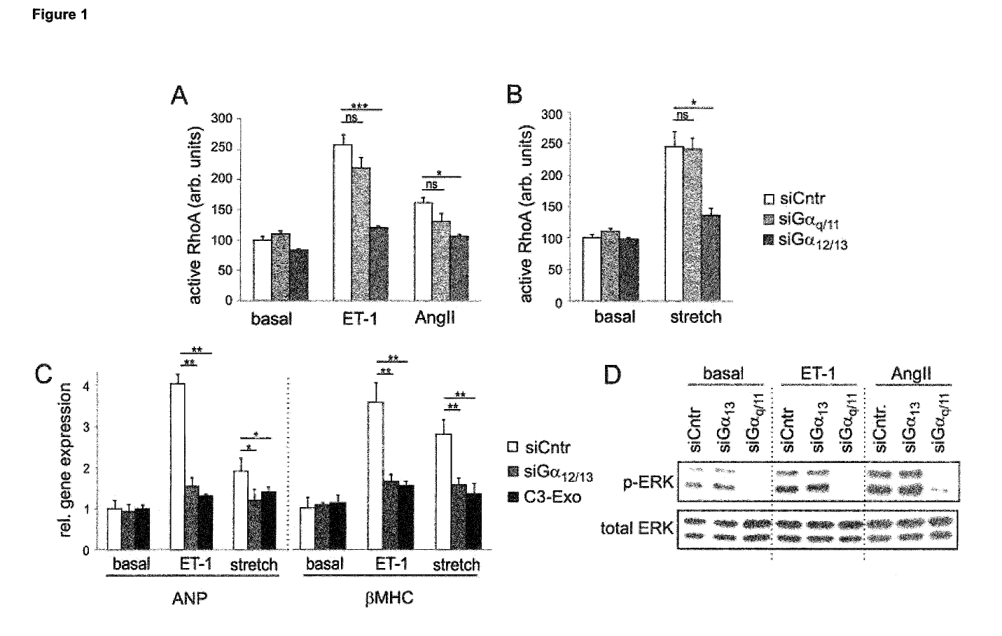

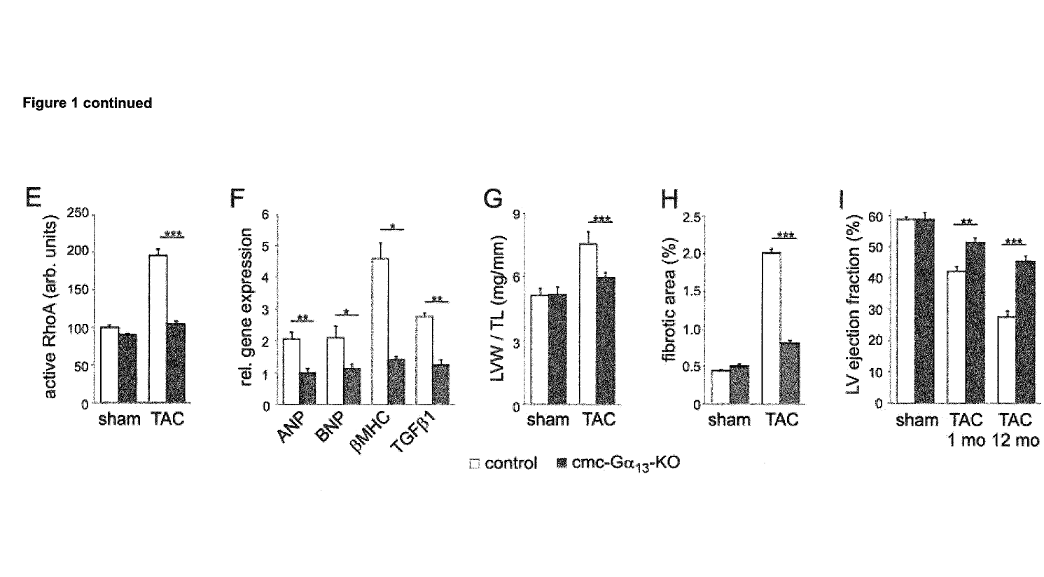

FIG. 1: Gα₇₃ is required for agonist-/stretch-induced RhoA activation and hypertrophic gene expression in neonatal rat ventricular myocytes (NRVM)

A,B, RhoA activation in siRNA-treated NRVM 3 min after application of GPCR agonists (A) or exposure to cyclic stretch (B) (n=4). C, qRT-PCR analysis of ANP and β-MHC expression in NRVM treated with siRNAs or pre-treated with RhoA inhibitor C3 exoenzyme (C3) 24 hours after application of ET-1 or exposure to cyclic stretch (n=4). D, After 3 min of agonist stimulation, protein extracts from siRNA-transfected NRVM were immunoblotted with antibodies directed against p42/44 ERK or total ERK. E, RhoA activation in left cardiac ventricles three days after TAC (n=3). F, Gene expression in isolated cardiomyocytes 5 days after TAC (n=3) (data presented as relative increase compared to sham-treated groups). G, Left ventricular weight/tibia length ratio (LVW/TL) 4 weeks after TAC (n=8-12). H, Fibrotic changes in left ventricles 4 weeks after TAC as determined by picrosirius red staining (n=6-8). I, Left ventricular (LV) ejection fraction (determined by MRI) before and 1 and 12 months after TAC (n=6). All qRT-PCR data are expressed relative to GAPDH expression. SiCntr, control siRNA; SiGα_{12/13/q/11}, siRNA directed against SiGα_{12/13/q/11}; *, p<0.05; **, p<0.01; ***, p<0.001; ns, not significant.

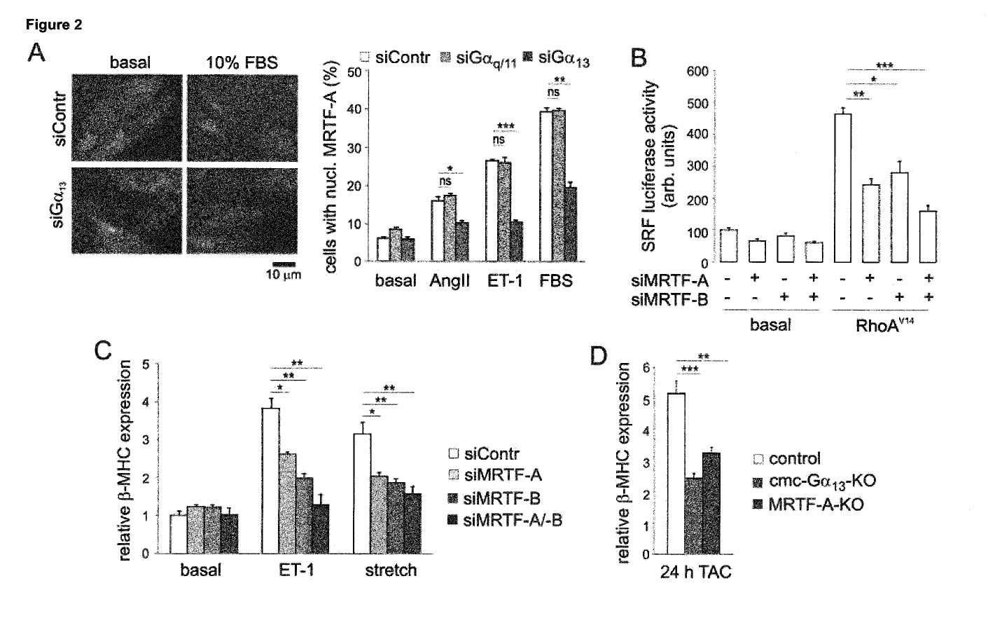

FIG. 2: Gα₁₃ regulates hypertrophic gene transcription through MRTFs

A, Translocation of myc-tagged MRTF-A in response to hypertrophic stimuli in siRNA-treated in H9c2 cells (n=3). B, Effect of RhoA^{V14} on SRF-luciferase activity in H9c2 cells after siRNA-mediated knockdown of MRTFs (n=3). C, Effect of siRNA-mediated knockdown of MRTFs on β-MHC expression in NRVM (n=3). D, β-MHC expression 24 h after TAC in cardiomyocytes from control mice (contr), cmc-Gα₁₃-KOs, and MRTF-A-deficient mice (MRTF-A-KO) (data presented as relative increase compared to sham) (n=3). FBS, fetal bovine serum; *, p<0.05; **, p<0.01; ***, p<0.001; ns, not significant.

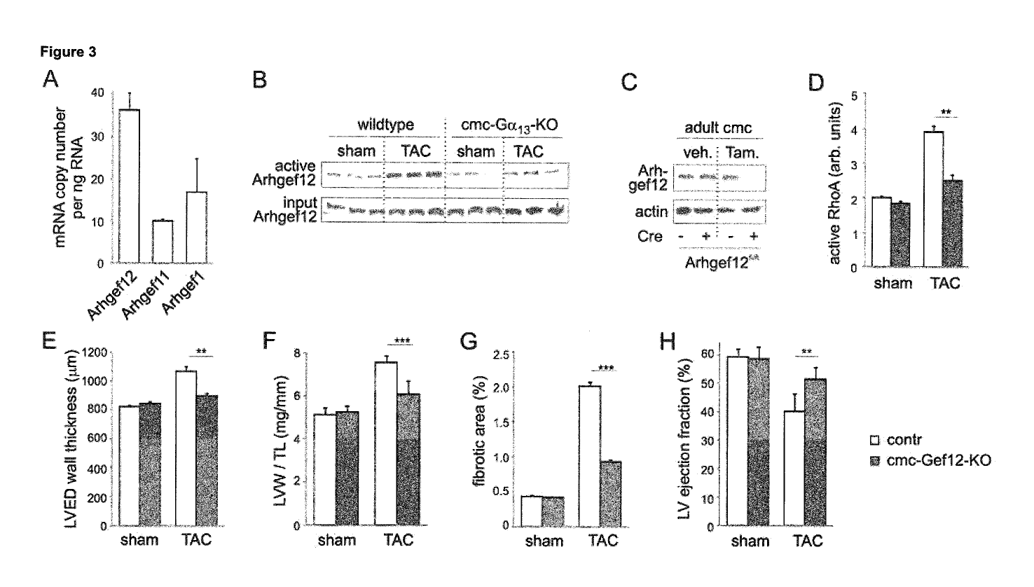

FIG. 3: Rhogef12 mediates Gα₁₃-dependent RhoA activation in hypertrophy

A, qRT-PCR analysis of RhoGEF expression in isolated cardiomyocytes from adult mice. B, Three days after TAC or sham surgery, active Rhogef12 was sedimented from lysed cardiac tissue with GST-RhoA^{G17A} and analyzed by Western blotting (n=3). C, Western blot analysis of RhoGEF12 protein levels in adult cardiomyocytes (cmc) from vehicle-/tamoxifen-treated Cre-negative or Cre-positive Gef12^{fl/fl} mice (actin as loading control). D, RhoA activation in adult hearts from control and mutant mice 3 days after TAC (n=3). E-H, Left ventricular end-diastolic (LVED) wall thickness (E), left ventricular weight/tibia length ratio (LVW/TL) (F), fibrotic area (G), and left ventricular (LV) ejection fraction (H) were determined by MRI (E, H) or postmortem analysis (F, G) 4 weeks after sham surgery or TAC (n=6-8). *, p<0.05; **, p<0.01; ***, p<0.001.

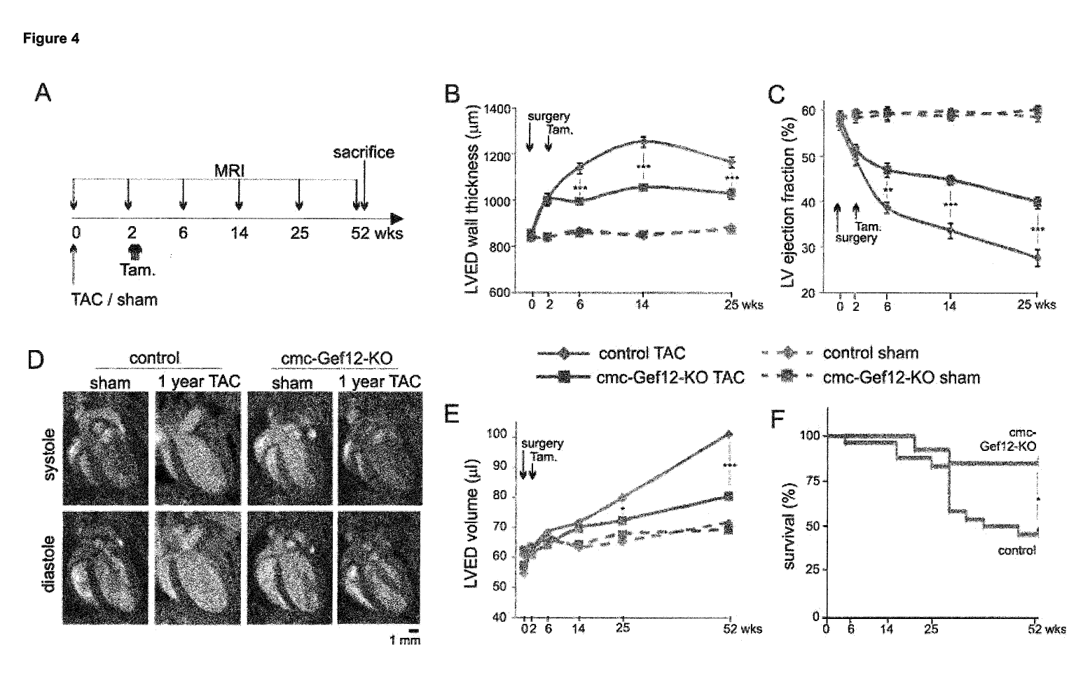

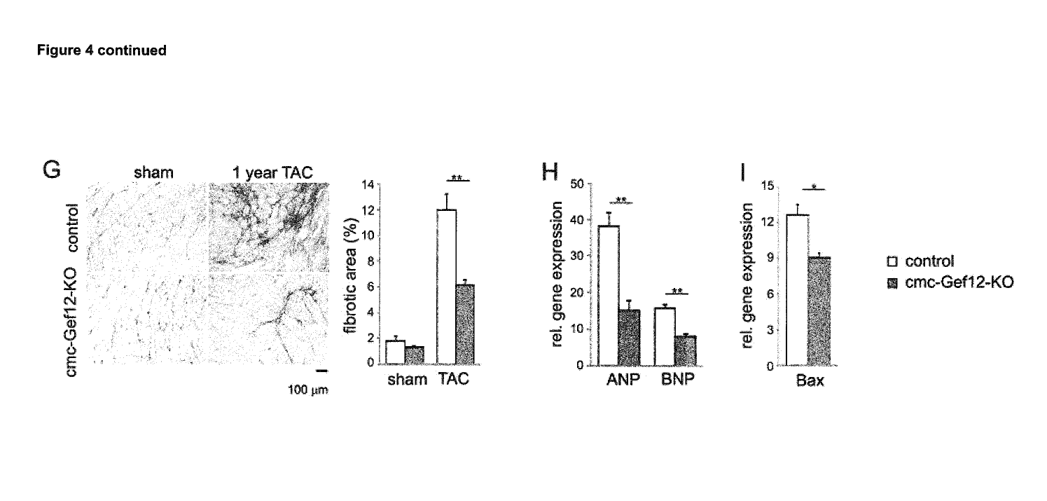

FIG. 4: Cardiomyocyte-specific inactivation of RhoGEF12 improves cardiac function and survival in mice with pre-existing hypertrophy

A, Experimental design. B-E, control mice (blue lines, diamonds) and not yet induced cmc-Gef12-KOs (red lines, squares) were subjected to sham surgery (hatched lines) or TAC (solid lines) at day 0, followed by tamoxifen-induction of recombination on days 14 to 18. MRI analysis of left ventricular end-diastolic (LVED) wall thickness (B), left ventricular (LV) ejection fraction (C), and LVED volume (D, exemplary MRI images, E, statistical evaluation) was performed before and 2, 6, 14, 25 and 52 weeks after surgery (n=15 per group). F, Survival plot for control and cmc-Gef12-KOs up to one year after TAC followed by tamoxifen injection (n=18-24). G, TAC-induced fibrosis (as determined by picrosirius red staining) 1 year after TAC (n=5). H, I, Expression of ANP, BNP (H) or Bax (I) one year after TAC as determined by qRT-PCR in whole hearts (data presented as relative increase compared to sham-treated group). LV, left ventricular; LVED, left ventricular end-diastolic; *, p<0.05; **, p<0.01; ***, p<0.001.

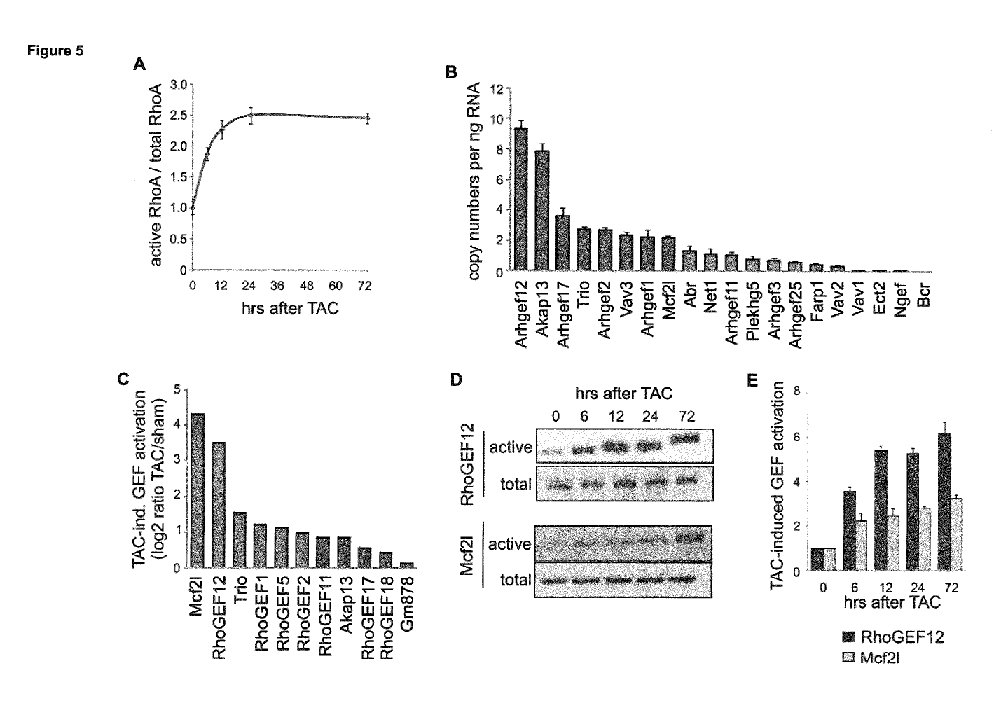

FIG. 5: Pressure overload-induced RhoGEF activation in vivo.

A, RhoA activation in wildtype hearts at different time points after TAC was determined by ELISA (n=6; data are expressed as a ratio “active RhoA/total RhoA” to correct for potential differences in RhoA expression, basal was set to 1). B, Quantitative RT-PCR analysis of RhoGEF expression in isolated adult murine cardiomyocytes (n=3). C, Activation of RhoGEF proteins 24 hours after TAC was determined by mass spectrometric analysis of proteins co-precipitated with beadcoupled nucleotide-free RhoA (data presented as log 2 of the LFQ intensity ratio between TAC operated sample and sham-operated sample). D, Activation of RhoGEF12 and Mcf2l was determined at different time points after TAC by precipitating RhoA-interacting proteins with bead-coupled nucleotide-free RhoA mutant as in C, followed by immunoblotting with antibodies directed against Mcf2l and RhoGEF12 (total cell lysate as loading control) (n=3). E, Statistical evaluation of D (basal set to 1). Hrs, hours; TAC, transverse aortic constriction.

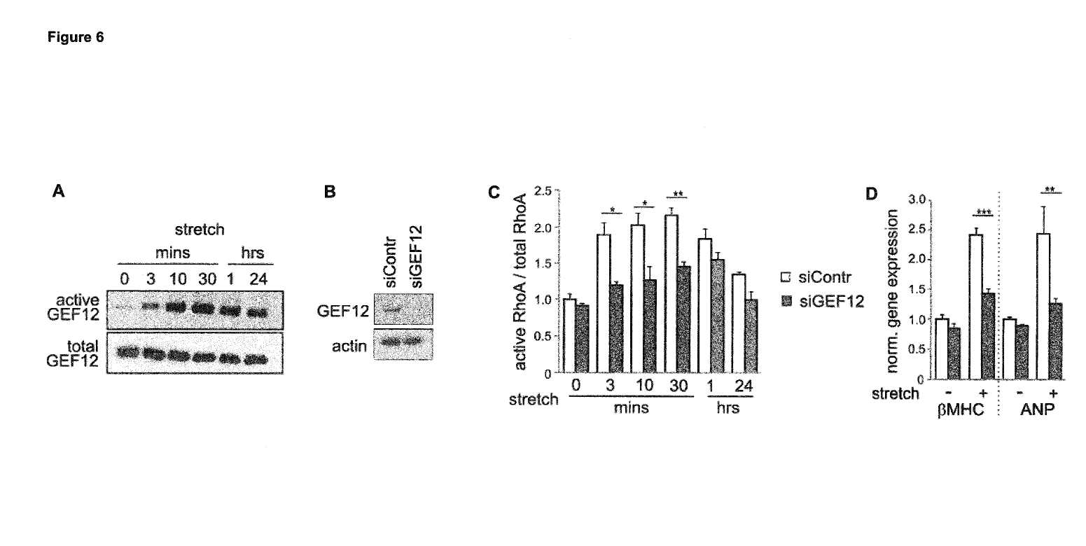

FIG. 6: Mechanism of stretch-induced RhoGEF12 activation in neonatal rat ventricular myocytes (NRVM).

A, Stretch-induced RhoGEF12 activation was determined by precipitating RhoA-interacting proteins with a bead-coupled nucleotide-free RhoA mutant, followed by immunoblotting with antibodies directed against RhoGEF12 (results representative of 3 independent experiments). B, Efficiency of RhoGEF12 knockdown in NRVMs as shown by immunoblotting with anti-RhoGEF12 antibodies (actin as loading control). C, RhoA activation in siRNA-treated NRVM at different time points after exposure to cyclic stretch (n=3; data are expressed as a ratio “active RhoA/total RhoA” to correct for potential differences in RhoA expression, basal was set to 1). D, Expression of hypertrophy-specific genes in siRNAtransfected NRVM was determined by quantitative RT-PCR after 24 hours of cyclic stretching (data normalized to GAPDH, control values set to 1).

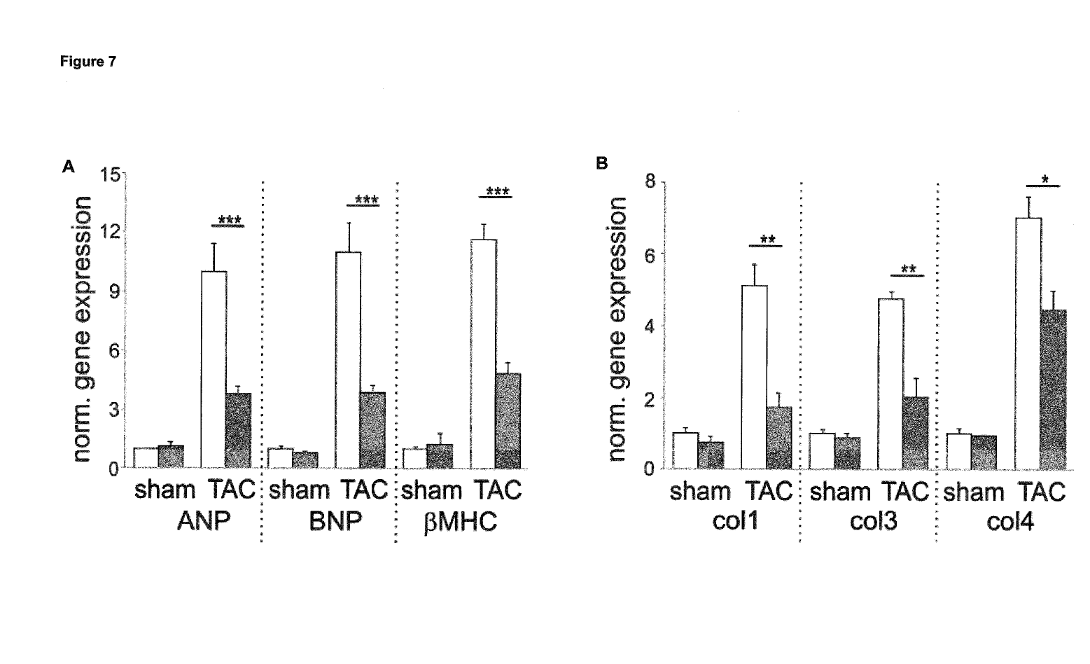

FIG. 7: RhoGEF12 mediates pressure overload-induced hypertrophy in vivo.

A, B, Expression of hypertrophy-specific genes (A) and pro-fibrotic genes (B) was determined by quantitative RT-PCR in hearts of control mice and cmc-GEF12-KOs four weeks after sham or TAC (n=4-6). Col1,3,4, collagen isoforms 1,3,4; *, p<0.05; **, p<0.01; ***, p<0.001.

The examples illustrate the invention:

### EXAMPLE 1

### siRNA-Mediated Knock-Down of G-Protein Subunits

To investigate the relative contribution of G_{12/13} and G_{q/11} to RhoA activation in vitro, siRNA-mediated knockdown of Gα_{q} and Gα₁₁, the two major α-subunits of the G_{q/11} family, as well as of the α-subunits of the G_{12/13} family, Gα₁₂ and Gα₁₃, was performed in neonatal rat ventricular myocytes (NRVM). The inventors found that knockdown of Gα_{12/13} reduced RhoA activation in response to GPCR agonists (FIG. 1A) or stretch (FIG. 1B), whereas knockdown of Gα_{q/11} had no significant effect. To investigate whether Gα_{12/13}-dependent RhoA activation was required for induction of cardiomyocyte hypertrophy, expression of hypertrophy-specific genes like atrial natriuretic peptide (ANP) and β-myosin heavy chain (β-MHC) was determined. While ET-1 or stretch increased gene expression in control cells, this response was strongly reduced by knockdown of Gα_{12/13} (FIG. 1C). The degree of reduction was the same as that observed after direct inhibition of RhoA by C3 exoenzyme, indicating that loss of RhoA activation underlies impaired hypertrophy gene expression in Gα_{12/13} knockdown cells (FIG. 1C). Both impaired RhoA activation and gene expression were mainly due to loss of Gα₁₃, while knockdown of Gα₁₂ had only minor effects. The effect of Gα₁₃ knockdown on hypertrophic gene expression was not due to an interference with the G_{q/11} pathway, since G_{q/11}-dependent phosphorylation of ERK1/2 (12) was normal in Gα₁₃-knockdown cells (FIG. 1D). These data suggest that Gα₁₃-dependent RhoA activation contributes to hypertrophic gene expression independently of Gα_{q/11}.

### EXAMPLE 2

### Role of Gα₁₃ In Vivo

To study the role of Gα₁₃-mediated RhoA activation in myocardial hypertrophy in vivo, mice with inducible, cardiomyocyte-specific deficiency for Gα₁₃ (cmc-Gα₁₃-KO) were generated. To do so, a new Cre-transgenic mouse line was established in which a fusion protein of the recombinase Cre and a tamoxifen-inducible estrogen receptor mutant (CreERT2) is expressed under control of the cardiomyocyte-specific α-myosin heavy chain (α-MHC) promoter. αMHC-CreERT2 mice were crossed to animals carrying a floxed version of Gna13 (13), the gene coding for Gα₁₃, and the efficiency of cardiomyocyte-specific gene inactivation was determined by Western blot analysis and RhoA activation assays. Magnetic resonance imaging (MRI) did not reveal any differences between the genotypes up to 6 months after induction. To induce hypertrophy in vivo, transverse aortic constriction (TAC) was used, which caused significant cardiac RhoA activation in control mice, but not in cmc-Gα₁₃-KOs (FIG. 1E). Five days after TAC, expression of ANP, BNP, β-MHC or TGF β 1 had increased in control cardiomyocytes, but not in Gα₁₃-deficient cardiomyocytes (FIG. 1F). Four weeks after TAC, control hearts showed significantly stronger hypertrophy than cmc-Gα₁₃-KOs in MRI and postmortem analysis of the left-ventricular weight/tibia length ratio (FIG. 1G). Importantly, loss of Gα₁₃-dependent hypertrophy resulted in reduced fibrosis (FIG. 1H) and improved ejection fraction at 1 and 12 months after pressure overloading (FIG. 1I). As an alternative model of murine cardiomyocyte hypertrophy the inventors used chronic AngII infusion, which promotes cardiac remodeling both through myocyte-autonomous mechanisms and induction of hypertension (14). Also in this model hypertrophy and fibrosis were significantly reduced in mutant mice, while ejection fraction was improved. Except for the loss of TAC-induced RhoA activation, all changes observed in cmc-Gα₁₃-KOs were quantitatively comparable to those observed in mice with tamoxifen-induced cardiomyocyte-specific Gα_{q/11}-deficiency.

### EXAMPLE 3

### Involvement of MRTFs

In order to understand how Gα₁₃-mediated RhoA activation was linked to induction of the hypertrophic gene program, the potential involvement of MRTFs was studied, which have been shown to translocate upon RhoA-mediated actin polymerization to the nucleus, where they act as co-activators of serum response factor (SRF)-dependent gene transcription (15, 16). The inventors found that various hypertrophic stimuli caused translocation of myc-tagged MRTF-A in cardiac H9c2 cells, and that this response was significantly impaired after knockdown of Gα₁₃, but not after knockdown of Gα_{q/11} (FIG. 2A). To investigate whether RhoA-dependent MRTF translocation contributed to transcriptional regulation, RhoA-induced expression of SRF-dependent genes after knockdown of MRTFs was studied. These experiments showed that overexpression of constitutively active RhoA^{V14} induced transcriptional activity of co-transfected SRF luciferase reporter constructs, and that this response was significantly impaired after knockdown of MRTF-A, the closely related MRTF-B, or both (FIG. 2B). Knockdown of MRTFs also strongly reduced ET-1- or stretch-induced upregulation of β-MHC expression in NRVM (FIG. 2C), indicating that MRTFs are crucial for the expression of hypertrophy genes in vitro. To verify these findings under in vivo conditions of pressure overload, the inventors analyzed early transcriptional changes induced by TAC in MRTF-A deficient mice (17), which have recently been shown to have impaired hypertrophic responses (18). It was found that MRTF-A-deficient cardiomyocytes showed 24 h after TAC the same impairment in β-MHC upregulation as Gα₁₃-deficient cardiomyocytes (FIG. 2D), suggesting that also in vivo a signaling cascade Gα₁₃-RhoA-MRTF controls hypertrophic gene expression in cardiomyocytes.

### EXAMPLE 4

### Activation of RhoA

Next, the mechanism by which Gα₁₃ activates RhoA under conditions of pressure overload was studied. Quantitative reverse transcription polymerase chain reaction (qRT-PCR) revealed that adult murine cardiomyocytes express various RhoGEF. Of the three and more RhoGEFs known to mediate G_{12/13}-dependent RhoA activation, Arhgef12, the gene encoding RhoGEF12, showed the strongest expression in adult murine cardiomyocytes (FIG. 3A, 5B). Also in human hearts RhoGEF12 is predominantly expressed. Affinity pulldown assays revealed that TAC enhanced RhoGEF12 activity in a Gα₁₃-dependent manner (FIG. 3B). More specifically, the RhoGEFs that showed strongest activation in response to TAC were Mcf2l and RhoGEF12, while other RhoGEFs did not show significantly increased RhoA-binding after TAC (FIG. 5C). This finding was confirmed by protein immunoblotting of left ventricular lysates obtained at different timepoints after TAC, demonstrating the strong and sustained activation of RhoGEF12, while activation of Mcf2l was less prominent (FIG. 5D, E). To investigate whether Gα₁₃ is also under in vivo conditions relevant for RhoGEF12 activation, TAC-induced RhoGEF12 activation was studied in mice with tamoxifen-inducible, cardiomyocyte-specific Gα₁₃ deficiency (cmc-Gα₁₃-KO). It was found that in the mutant mouse line activation of RhoGEF12 was significantly reduced 24 hours after TAC, indicating that also under conditions of pressure overload Gα_{12/13} is crucial for RhoGEF12 activation. To study the role of RhoGEF12 in hypertrophy in vivo, tamoxifen-inducible, cardiomyocyte-specific RhoGEF12-deficient animals (cmc-Gef12-KO) (FIG. 3C) were generated. No changes in basal heart function were observed after tamoxifen treatment. Upon pressure overloading by TAC, RhoA activation was strongly reduced in cmc-Gef12-KOs (FIG. 3D), as was TAC-induced hypertrophy (FIG. 3E, F) and fibrosis (FIG. 3G). As in cmc-Gα₁₃-KOs, ejection fraction was preserved compared to wildtype mice (FIG. 3H). Four weeks after TAC the expression of hypertrophy-specific genes (FIG. 7A) was significantly reduced in mutant mice. Also, cmc-GEF12-KOs showed lower expression of collagen isoforms (FIG. 7B).

### EXAMPLE 5

### RhoGEF12 Inhibition in Established Hypertrophy

Finally, it was investigated whether inhibition of the Gα₁₃/RhoGEF12-mediated signaling pathway would also prove beneficial if applied in already established hypertrophy. To study this, TAC or sham surgery was performed two weeks prior to tamoxifen-mediated induction of RhoGEF12-deficiency, and cardiac performance was followed by MRI for up to one year (FIG. 4A). During the first two weeks after surgery, both control animals and not yet induced RhoGEF12 mutants showed similar increases in left ventricular wall thickness and deterioration of ejection fraction (FIG. 4B, C). When recombination was induced by tamoxifen injection two weeks after surgery, deficient mice were protected from further increases in wall thickness, and ejection fraction was preserved compared to controls. Interestingly, at about 6 months after TAC wildtype mice, but not cmc-Gef12-KOs started to show progressive ventricular dilation (FIG. 4D, E), resulting in significantly increased mortality (FIG. 4F). Hearts of surviving control mice showed 1 year after TAC significantly stronger fibrosis (FIG. 4G), elevated expression of heart failure markers ANP and BNP (FIG. 4H), and increased transcription of pro-apoptotic genes such as Bax (FIG. 4I).

### EXAMPLE 6

### Materials and Methods

### 6.1 Materials and Chemicals

Angiotensin II, endothelin-1, and tamoxifen were purchased from Sigma-Aldrich (St. Louis, Mo., USA). C3-Exoenzyme (Cell permeable Rho inhibitor (CT03)) was from Cytoskeleton (Denver, Colo., USA). Antibodies to Gα₁₃ (sc-26788), Gα_{q/11} (sc-392), RhoGEF12 (sc-15439), MRTF-A (sc-21558), MRTF-B (sc-47282), p-ERK (sc-7383), c-Myc (sc-40), and β-actin (sc-47778) were from Santa Cruz Biotechnology (Santa Cruz, Calif., USA), antibodies to total ERK (137F5) were from Cell Signaling (Beverly, Mass., USA). The plasmid of MRTF-A was a kind gift from Dr. Nakano (Juntendo University, Japan). The plasmids of RhoA mutants were a kind gift from Dr. Kaibuchi (Nagoya University, Japan).

### 6.2 Generation and Characterization of αMHC-CreERT2 Mice

To generate tamoxifen-inducible cardiomyocyte-specific Cre-transgenic mice, a cassette consisting of the CreERT2 cDNA followed by a polyadenylation signal from bovine growth hormone and a module containing the β-lactamase gene flanked by frt sites³¹ was introduced into the start codon of the mouse α-MHC (MYH6) gene (Accession No: NM_{—}001164171.1) carried by BAC RP23-93K3 (from Chori BACPAC Resources Center) using RedE/T-mediated recombination³². Correctly targeted recombinants were verified by Southern blotting and PCR. After FLPe-mediated recombination, the recombined BAC was injected into male pronuclei derived from fertilized FvB/N oocytes. Transgenic offspring was analyzed for BAC insertion by PCR. To verify the inducibility and specificity of the Cre fusion protein, the inventors mated αMHC-CreERT2 mice with the Cre-reporter line Gt(ROSA)26Sortm1sor (ROSA26-LacZ) (obtained from the Jackson Laboratories, Bar Harbor, Me., USA). Double transgenic progeny were treated with daily intraperitoneal injections of 1 mg tamoxifen for five consecutive days or vehicle alone and sacrificed one or two weeks after the end of induction. Histological analysis of β-galactosidase activity was performed on 12 μm cryosections according to standard protocols. Genotyping of transgenic animals was done using primers P1 (5′-CTTACCCCACATAGACCTCTGACA-3′) (SEQ ID NO: 1) and P2 (5′-TGCTGTTGGATGGTCTTCACAG-3′) (SEQ ID NO: 2).

### 6.3 Experimental Animals

Gna13^{fl/fl}; 12^{-/-} mice, Arhgef12^{fl/fl} mice, and MRTF-A-deficient mice have been reported previously³³⁻³⁵. Cre-mediated recombination of floxed alleles was induced by intraperitoneal injection of 1 mg tamoxifen dissolved in 50 ul of Miglyol on five consecutive days. Vehicle treated mice received Miglyol only. For in vivo experiments, tamoxifen-treated αMHC-CreERT2^{+/-}; Gna13^{wt/wt}; Arhgef12_{wt/wt} mice were used as controls. Experiments were usually performed one or two weeks after the end of induction.

### 6.4 Surgical Interventions: Transverse Aortic Constriction, Osmotic Minipumps

For transverse aortic constriction (TAC), male mice of an age of 8-10 weeks were anesthetized with pentobarbital sodium (50 mg/kg). The chest was opened and the aortic arch was identified. TAC was created by ligating the transverse aorta between the right innominate and the left common carotid artery against a blunted 24-gauge needle using a 7-0 suture³⁶. The needle was then gently retracted. The sham procedure was identical except that the aorta was not ligated. For the implantation of osmotic minipumps, male mice of an age of 12-14 weeks were anesthetized with pentobarbital sodium and osmotic minipumps releasing AngII (150 ng g⁻¹ h⁻¹) (Alzet, Cupertino, Calif., USA) were implanted subcutaneously in the back region where they remained for 4 weeks.

### 6.5 Magnetic Resonance Imaging

Cardiac MRI measurements were performed on a 7.0 T Bruker Pharmascan, equipped with a 300 mT/m gradient system, using a custom-built circularly polarized birdcage resonator and the Early Access Package for self-gated cardiac Imaging (Bruker, Ettlingen, Germany)³⁷. The mice were measured under volatile isoflurane (2.0%) anesthesia. The measurement is based on the gradient echo method (repetition time=6.2 ms; echo time=1.6 ms; field of view=2.20×2.20 cm; slice thickness=1.0 mm; matrix=128×128; repetitions=100). The imaging plane was localized using scout images showing the 2- and 4-chamber view of the heart, followed by acquisition in short axis view, orthogonal on the septum in both scouts. Multiple contiguous short-axis slices consisting of 9 or 10 slices were acquired for complete coverage of the left ventricle. MRI data were analyzed using Qmass digital imaging software (Medis, Leiden, Netherlands).

### 6.6 Isolation of Neonatal Rat Ventricular Cardiomyocytes

Neonatal rat ventricular myocytes (NRVM) were isolated from 1-2 days old rat neonates using a kit from Worthington Biochemical Corporation (Lakewood, N.J., USA). After digestion, cells were pre-plated for 1 h to remove nonmyocytes, plated on cell culture dishes pre-coated with 1% gelatin (Sigma-Aldrich) and then cultured in Dulbecco's modified Eagle's medium (DMEM) with 10% fetal bovine serum. The following day, cells were cultured in serum-free DMEM medium containing 100 μM 5-bromo 2′-deoxy-uridine (BrdU; Sigma-Aldrich). NRVM were transfected with siRNAs (Qiagen, Chatsworth, Calif., USA or Sigma-Aldrich) using Lipofectamine RNAiMAX (Invitrogen, San Diego, Calif., USA) 3 h and 20 h after plating according to the manufacturer's instructions. The following siRNA target sequences were used: Gα₁₃: 5′-CAGCAACGTGATCAAAGGTAT-3′ (SEQ ID NO: 3); Gα₁₂: 5′-CCGCGACACCATCTTCGACAA-3′ (SEQ ID NO: 4); Gα_{q}: 5′-AAGCACTCTTTAGAACCATTA-3′ (SEQ ID NO: 5); Gα₁₁: 5′-CACAACTGGCATCATCGAGTA-3′ (SEQ ID NO: 6); Arhgef12: 5′-GTCTCAAGTTGTCTGAGTA-3′ (SEQ ID NO: 7); Mkl1 (MRTF-A): 5′-CAATTTGCCTCCACTTAGT-3′ (SEQ ID NO: 8); Mkl2 (MRTF-B): 5′-CTTAGAACCTGTGAACAGT-3′ (SEQ ID NO: 9); Arhgef12-II: 5′-CCAAGTATTCTATCAGCGA-3′ (SEQ ID NO: 37); Gα₁₃-II: 5′-CAGTATCTTCCTGCTATAAGA-3′ (SEQ ID NO: 38); Gα₁₂-II: 5′-GTGAGTCAGTGAAGTACTT-3′ (SEQ ID NO: 39); CD44: 5′-CTACTTCACTGGAAGGCTA-3′ (SEQ ID NO: 40); FYN: 5′-GAGAATCCCTGCAGTTGAT-3′ (SEQ ID NO: 41); SRC: 5′-CAGCTTGTGGCTTACTACT-3′ (SEQ ID NO: 42); RhoA: 5′-CAGACACTGATGTTATACT-3′ (SEQ ID NO: 43).

### 6.7 Isolation of Adult Mouse Left Ventricular Cardiomyocyte

Adult mouse cardiomyocyte were isolated as previously described³⁸. Briefly, the heart was removed quickly and cannulated from the aorta with a blunted 27G needle to allow retrograde perfusion of the coronary arteries. The heart was first washed with 50 ml of perfusion buffer (113 mM NaCl, 4.7 mM KC, 0.6 mM KH₂PO₄, 1.2 mM MgSO₄, 12 mM NaHCO₃, 10 mM KHCO₃, 10 mM HEPES, 30 mM Taurine, 10 mM 2,3-Butanedione monoxime, 5.5 mM Glucose, pH 7.46), then digested with 75 ml of digesting buffer (perfusion buffer with 0.05 mg/ml Liberase DH (Roche, Mannheim, Germany) and 12.5 μM CaCl₂). The heart was removed from the perfusion apparatus and the left ventricle was minced in digesting buffer. The calcium concentration was slowly increased from 12.5 μM to 1 mM. Undissociated clumps were removed by filtration through 100 μm nylon mesh. Centrifugation (50×g, 1 min) was performed 3 times to enrich cardiomyocytes. The cardiomyocytes were seeded on laminin-coated dishes (2 μg laminin/cm²) after the last centrifugation. To collect non-cardiomyocyte cells, the supernatant after the first centrifugation was seeded on uncoated dishes. Two hours after seeding, attached cells were collected as non-cardiomyocyte cells.

### 6.8 Cell Culture

For luciferase assays, H9c2 cells cultured in 10-cm dishes were transfected with siRNA using Lipofectamine RNAiMAX 24 h and 48 h after plating. Four hours after the second siRNA transfection, cells were transfected with pGL4.34-luc2P/SRF-RE (5 μg, Promega, Madison, Wis., USA) and HA-RhoA plasmids (15 μg) using Lipofectamine 2000 (Invitrogen). Four hours after plasmid transfection, the growth medium was replaced with serum-free medium. After a 40-h serum-free incubation, cells were washed with phosphate-buffered saline (PBS) twice and collected in PBS using a cell scraper. The samples were centrifuged and the supernatants were discarded. Luciferase activities were measured using a luciferase assay system (Promega). To determine MRTF-A translocation, H9c2 cells were transfected with pcDNA3-myc MRTF-A using Lipofectamine 2000. The cells were incubated in serum-free medium for 24 h before being treated with 1 μM ET-1, Angll, or 10% fetal bovine serum for 1 h and fixed with 4% formaldehyde in PBS. The subcellular distribution of myc-tagged MRTF-A was determined by immunostaining with an anti-myc antibody. Stretch-induced changes in cardiomyocyte size were quantified using NIH ImageJ software; 75 cell were evaluated per experimental condition. To study the cellular effects of direct stimulation of G12/13 signaling, NRVM were seeded on 6-well tissue culture plates without coating (Greiner Bio-One, Germany). NRVM were incubated in serum-free medium for 18 h and then incubated in 500 μl of serum-free medium containing IgG or endothelin-1 (1 μM) for 5 min. After 5 min incubation, RhoGEF pull-down assay was performed. To study stretch-induced MRTF-A translocation, isolation of nuclear proteins and cytoplasmic proteins extraction was performed with DUALXtract (Dualsystems, Switzerland).

### 6.9 mRNA Expression Analysis

RNA was extracted from left ventricles with the RNA fibrosis tissue kit (Qiagen) and from NRVM or adult mouse cardiomyocytes with the RNeasy Mini kit (Qiagen) according to the manufacture's protocol. In some cases, NRVM were pretreated with C3-Exoenzyme for 4 h at a concentration of 1 μg/ml. Reverse transcriptase (RT) reaction was performed using the QuantiTect Reverse Transcription kit (Qiagen). Quantitative RT-PCR was performed using the LightCycler 480 Probe Master or LightCycler 480 SYBR Green Master (Roche). Genomic DNA from mouse tails was used as a universal standard to calculate gene copy number of Arhgef1, Arhgef11 and Arhgef12³⁹. The following primers were used:

| | |
|---|---|
| rat Ga₁₂ | |
| 5′-CGGCAAGTCCACCTTCCTCAAGC-3′ | (SEQ ID NO: 10)/ |
| 5′-TGGTGTCGCGGAACTCCAGCA-3′; | (SEQ ID NO: 11) |
| rat β-MHC | |
| 5′-GAGGGCGGACATTGCCGAGT-3′ | (SEQ ID NO: 12)/ |
| 5′-AAGGCTCCAGGTCTCAGGGCTTC-3′; | (SEQ ID NO: 13) |
| rat GAPDH | |
| 5′-GGCGGCTTCGGGGCACATTT-3′ | (SEQ ID NO: 14)/ |
| 5′-GGGCCAGGCAGTTGGTGGTAC-3′; | (SEQ ID NO: 15) |
| mouse ANP | |
| 5′-CACAGATCTGATGGATTTCAAGA-3′ | (SEQ ID NO: 16)/ |
| 5′-CCTCATCTTCTACCGGCATC-3′; | (SEQ ID NO: 17) |
| mouse BNP | |
| 5′-GTCAGTCGTTTGGGCTGTAAC-3′ | (SEQ ID NO 18)/ |
| 5′-AGACCCAGGCAGAGTCAGAA-3′; | (SEQ ID NO: 19) |
| mouse β-MHC | |
| 5′-CGCATCAAGGAGCTCACC-3′ | (SEQ ID NO: 20)/ |
| 5′-CTGCAGCCGCAGTAGGTT-3′; | (SEQ ID NO: 21) |
| mouse TGFβ1 | |
| 5′-TGGAGCAACATGTGGAACTC-3′ | (SEQ ID NO: 22)/ |
| 5′-CAGCAGCCGGTTACCAAGACCAAG-3′; | (SEQ ID NO: 23) |
| mouse Collagen 1 | |
| 5′-CATGTTCAGCTTTGTGGACCT-3′ | (SEQ ID NO: 24)/ |
| 5′-GCAGCTGACTTCAGGGATGT-3′; | (SEQ ID NO: 25) |
| mouse Collagen 3 | |
| 5′-TCCCCTGGAATCTGTGAATC-3′ | (SEQ ID NO: 26)/ |
| 5′-TGAGTCGAATTGGGGAGAAT-3′; | (SEQ ID NO: 27) |
| mouse Collagen 4 | |
| 5′-TTAAAGGACTCCAGGGACCAC-3′ | (SEQ ID NO: 28)/ |
| 5′-CCCACTGAGCCTGTCACAC-3′; | (SEQ ID NO: 29) |
| mouse BAX | |
| 5′-GTGAGCGGCTGCTTGTCT-3′ | (SEQ ID NO: 30)/ |
| 5′-GGTCCCGAAGTAGGAGAGGA-3′; | (SEQ ID NO: 31) |
| mouse GAPDH | |
| 5′-AGCTTGTCATCAACGGGAAG-3′ | (SEQ ID NO: 32)/ |
| 5′-TTTGATGTTAGTGGGGTCTCG-3′; | (SEQ ID NO: 33) |

Primers for the analysis of RhoGEF expression were used as described previously⁴⁰. Data are in all cases presented after normalization to GAPDH, in some cases the relative change compared to basal conditions was calculated.

### 6.10 Determination of Activated RhoA

NRVM or mouse cardiomyocytes were incubated in serum-free medium for 18 h and then treated with 1 μM ET-1 or AngII for 3 minutes or stretched by 10% for 5 minutes with a frequency of 1 Hz using a Flexcell system (Flexcell, Hillsborough, N.C., USA). Activated RhoA was measured by G-LISA RhoA activation Assay kit (Cytoskeleton) using 0.5 mg/ml protein per sample.

### 6.11 Determination of Activated RhoGEF12

Determination of activated RhoGEF12 was performed as described previously⁴¹ with the following modifications: Three days after surgery, hearts were extirpated and frozen in liquid nitrogen. The whole hearts were disrupted by a homogenizer in lysis buffer (0.2% TritonX-100, 20 mM HEPES, pH7.5, 150 mM NaCl, 5 mM MgCl₂, protease inhibitors), and the protein concentration of the supernatants was determined after centrifugation by Precision Red Advanced Protein Assay Reagent (Cytoskeleton). Samples containing 1.5 mg of total protein were incubated at 4° C. for 1 h with 20 μg GST-RhoA^{G17A} bound to Glutathione-Sepharose 4B beads. The beads were washed 4 times with lysis buffer and then eluted with SDS sample buffer. The eluates were subjected to immunoblot analysis with an anti-RhoGEF12 antibody.

### 6.12 Histological Analyses

Freshly dissected heart tissue was fixed in 4% paraformaldehyde, dehydrated and embedded in paraffin. Embedded hearts were stained with picrosirius red according to standard protocol. Twenty randomly chosen frames from the sections were quantified to assess the degree of heart fibrosis using NIH ImageJ software.

### 6.13 Western Blotting

Samples were subjected to sodium dodecyl sulfate polyacrylamide gel electrophoresis (SDS-PAGE), transferred to nitrocellulose transfer membranes (Whatman, Dassel, Germany), and then incubated with primary antibodies as indicated. Equal loading was checked by antibody to α-tublin or actin.

### 6.14 Statistical Analyses

Data are presented as means±standard errors of the means (SEM). Comparisons between two groups were performed with unpaired student's t-test, comparisons between more than two groups by ANOVA followed by Bonferroni post hoc test. Comparisons between more than two groups at different time points were done by repeated measures ANOVA followed by Bonferroni post hoc test. “n” refers to the number of independent experiments or mice per group.

### EXAMPLE 7

### RhoGEF12-Dependent Rhoa Activation in Cardiomyocyte Hypertrophy

In order to investigate the role of RhoGEF12-dependent RhoA activation in cardiomyocyte hypertrophy, we studied stretch-induced effects in cultured neonatal rat ventricular myocytes (NRVM) in vitro. Mechanical stress induced a fast and stable activation of RhoGEF12 (FIG. 6A) and RhoA (FIG. 6C) with a maximal response between 3 and 30 minutes. SiRNA-mediated knockdown of RhoGEF12 (FIG. 6B) strongly reduced stretch-induced RhoA activation (FIG. 6C) as well as expression of hypertrophy-specific genes such as β-myosin heavy chain (βMHC) or atrial natriuretic peptide (ANP) (FIG. 6D). Also stretch-induced increases in cell size were significantly reduced after knockdown of RhoGEF12 compared to control cells. To investigate whether loss of RhoGEF12-dependent RhoA activation was sufficient to explain reduced hypertrophic response to stretch, NRVM were pretreated with the RhoA inhibitor C3 exoenzyme or siRNA directed against RhoA. Both C3 exoenzyme and knockdown of RhoA fully mimicked the effect of RhoGEF12 knockdown, indicating that RhoGEF12 indeed controls hypertrophic gene expression through RhoA activation. The role of potential activators of RhoGEF12 such as G_{12/13} (52), G_{q/11} (53), or CD44 (54) was studied next. SiRNA-mediated knockdown in NRVM revealed that inactivation of the α-subunits of the G_{12/13} family (Gα_{12/13}) time-dependently reduced stretch-induced RhoGEF12 activation, and comparable effects were observed on the level of RhoA activation. In contrast, knockdown of the α-subunits of the G_{q/11} family (Gα_{q/11}), CD44 was without effect (data not shown).

### REFERENCES

1. J. A. Hill, E. N. Olson, N Engl J Med 358, 1370 (Mar. 27, 2008).
2. N. Frey, E. N. Olson, Annu Rev Physiol 65, 45 (2003).
3. J. Heineke, J. D. Molkentin, Nat Rev Mol Cell Biol 7, 589 (August, 2006).
4. H. A. Rockman, W. J. Koch, R. J. Lefkowitz, Nature 415, 206 (Jan. 10, 2002).
5. G. W. Dorn, 2nd, H. S. Hahn, Ann N Y Acad Sci 1015, 225 (May, 2004).
6. S. Miyamoto et al., J Cardiovasc Transl Res 3, 330 (August, 2010).
7. A. Clerk, P. H. Sugden, Circ Res 86, 1019 (May 26, 2000).
8. K. Burridge, K. Wennerberg, Cell 116, 167 (Jan. 23, 2004).
9. S. A. Akhter et al., Science 280, 574 (Apr. 24, 1998).
10. N. Wettschureck et al., Nat Med 7, 1236 (November, 2001).
11. S. Fukuhara, H. Chikumi, J. S. Gutkind, Oncogene 20, 1661 (Mar. 26, 2001).
12. J. D. Molkentin, G. W. Dorn, 2nd, Annu Rev Physiol 63, 391 (2001).
13. A. Moers et al., Nat Med 9, 1418 (November, 2003).
14. K. D. Schluter, S. Wenzel, Pharmacol Ther 119, 311 (September, 2008).
15. E. N. Olson, A. Nordheim, Nat Rev Mol Cell Biol 11, 353 (May, 2010).
16. G. Posern, R. Treisman, Trends Cell Biol 16, 588 (November, 2006).
17. Y. Sun et al., Mol Cell Biol 26, 5809 (August, 2006).
18. K. Kuwahara et al., Mol Cell Biol 30, 4134 (September, 2010).
19. S. G. Finn, S. G. Plonk, S. J. Fuller, Cardiovasc Res 42, 140 (April, 1999).
20. Y. Maruyama et al., Circ Res 91, 961 (Nov. 15, 2002).
21. M. Nishida et al., EMBO J 27, 3104 (Dec. 3, 2008).
22. J. Sadoshima, Y. Xu, H. S. Slayter, S. Izumo, Cell 75, 977 (Dec. 3, 1993).
23. T. Yamazaki et al., J Biol Chem 271, 3221 (Feb. 9, 1996).
24. Y. Zou et al., Nat Cell Biol 6, 499 (June, 2004).
25. G. W. Dorn, 2nd, Cardiovasc Res 81, 465 (Feb. 15, 2009).
26. V. P. Sah et al., J Clin Invest 103, 1627 (June, 1999).
27. D. P. Del Re, S. Miyamoto, J. H. Brown, J Biol Chem 282, 8069 (Mar. 16, 2007).
28. R. Kakkar, R. T. Lee, Circ Res 106, 47 (Jan. 8, 2010).
29. E. M. Small et al., Circ Res 107, 294 (Jul. 23, 2010).
30. X. Zhang et al., Am J Physiol Heart Circ Physiol 280, H1782 (April, 2001).
31. Fell, R., et al. Ligand-activated site-specific recombination in mice. Proc Natl Acad Sci USA 93, 10887-10890 (1996).
32. Muyrers, J. P., Zhang, Y., Testa, G. & Stewart, A. F. Rapid modification of bacterial artificial chromosomes by ET-recombination. Nucleic Acids Res 27, 1555-1557 (1999).
33. Sun, Y., et al. Acute myeloid leukemia-associated Mkl1 (Mrtf-a) is a key regulator of mammary gland function. Mol Cell Biol 26, 5809-5826 (2006).
34. Moers, A., et al. G13 is an essential mediator of platelet activation in hemostasis and thrombosis. Nat Med 9, 1418-1422 (2003).
35. Herroeder, S., et al. Guanine nucleotide-binding proteins of the G12 family shape immune functions by controlling CD4+ T cell adhesiveness and motility. Immunity 30, 708-720 (2009).
36. Rockman, H. A., et al. Segregation of atrial-specific and inducible expression of an atrial natriuretic factor transgene in an in vivo murine model of cardiac hypertrophy. Proc Natl Acad Sci USA 88, 8277-8281 (1991).
37. Larson, A. C., et al. Self-gated cardiac cine MRI. Magn Reson Med 51, 93-102 (2004).
38. Zhou, Y. Y., et al. Culture and adenoviral infection of adult mouse cardiac myocytes: methods for cellular genetic physiology. Am J Physiol Heart Circ Physiol 279, H429-436 (2000).
39. Yun, J. J., et al. Genomic DNA functions as a universal external standard in quantitative real-time PCR. Nucleic Acids Res 34, e85 (2006).
40. Wirth, A., et al. G12-G13-LARG-mediated signaling in vascular smooth muscle is required for salt-induced hypertension. Nat Med 14, 64-68 (2008).
41. Garcia-Mata, R., et al. Analysis of activated GAPs and GEFs in cell lysates. Methods Enzymol 406, 425-437 (2006).
42. Kourlas, P. J., Strout, M. P., Becknell, B., Veronese, M. L., Croce, C. M., Theil, K. S., Krahe, R., Ruutu, T., Knuutila, S., Bloomfield, C. D., and Caligiuri, M. A. 2000. Identification of a gene at 11q23 encoding a guanine nucleotide exchange factor: evidence for its fusion with MLL in acute myeloid leukemia. Proc Natl Acad Sci USA 97 (5):2145-2150.
43. Kozasa, T., Hajicek, N., Chow, C. R., and Suzuki, N. 2011. Signalling mechanisms of RhoGTPase regulation by the heterotrimeric G proteins G12 and G13. J Biochem 150 (4):357-369.
44. Riobo, N. A., and Manning, D. R. 2005. Receptors coupled to heterotrimeric G proteins of the G12 family. Trends Pharmacol Sci 26 (3):146-154.
45. Strathmann, M. P., and Simon, M. I. 1991. G alpha 12 and G alpha 13 subunits define a fourth class of G protein alpha subunits. Proc Natl Acad Sci USA 88 (13):5582-5586.
46. Suzuki, N., Tsumoto, K., Hajicek, N., Daigo, K., Tokita, R., Minami, S., Kodama, T., Hamakubo, T., and Kozasa, T. 2009. Activation of leukemia-associated RhoGEF by Galpha13 with significant conformational rearrangements in the interface. J Biol Chem 284 (8):5000-5009.
47. Worzfeld, T., Wettschureck, N., and Offermanns, S. 2008. G(12)/G(13)-mediated signalling in mammalian physiology and disease. Trends Pharmacol Sci 29 (11):582-589.
48. Olson E N & Nordheim A, Nat Rev Mol Cell Biol 11: 353-365, 2010.
49. Aoki et al., Circ Res 82:666-676, 1998;
50. Hoshijima et al., J Biol Chem 273:7725-7730, 1998.
51. Guilluy, C., V. Swaminathan, R. Garcia-Mata, E. T. O'Brien, R. Superfine, and K. Burridge. 2011. The Rho GEFs LARG and GEF-H1 regulate the mechanical response to force on integrins. Nat Cell Biol. 13:722-7.
52. Fukuhara, S., H. Chikumi, and J. S. Gutkind. 2000. Leukemia-associated Rho guanine nucleotide exchange factor (LARG) links heterotrimeric G proteins of the G(12) family to Rho. FEBS Lett. 485:183-8.
53. Booden, M. A., D. P. Siderovski, and C. J. Der. 2002. Leukemia-associated Rho guanine nucleotide exchange factor promotes G alpha q-coupled activation of RhoA. Mol Cell Biol. 22:4053-61.
54. Bourguignon, L. Y., E. Gilad, A. Brightman, F. Diedrich, and P. Singleton. 2006. Hyaluronan-CD44 interaction with leukemia-associated RhoGEF and epidermal growth factor receptor promotes Rho/Ras co-activation, phospholipase C epsilon-Ca2+ signaling, and cytoskeleton modification in head and neck squamous cell carcinoma cells. J Biol Chem. 281:14026-40.
55. Gu J L, Muller S, Mancino V, Offermanns S, Simon M I. Interaction of G alpha(12) with G alpha(13) and G alpha(q) signaling pathways. Proc Natl Acad Sci USA. 2002; 99:9352-9357

1. (canceled)

2. A method of preventing and/or treating heart failure associated with cardiac hypertrophy and/or cardiac fibrosis and diseases associated therewith comprising administering a pharmaceutically effective amount of an inhibitor of Rho guanine nucleotide exchange factor 12 (RhoGEF12) or an inhibitor of an activator of RhoGEF12 to a subject in need thereof.

3. The method of claim 2, wherein the inhibitor is an antibody or a fragment or derivative thereof, an aptamer, an siRNA, an shRNA, a miRNA, a ribozyme, an antisense nucleic acid molecule, a small molecule or modified versions of these inhibitors.

4. The method of claim 2 or 3, wherein the activator of RhoGEF12 is the G-protein alpha subunit Gα₁₃.

5. A method of identifying an inhibitor of Rho guanine nucleotide exchange factor 12 (RhoGEF12) or an inhibitor of an activator of RhoGEF12 suitable as a lead compound and/or as a medicament for the prevention and/or treatment of heart failure associated with cardiac hypertrophy and/or cardiac fibrosis and diseases associated therewith, comprising the steps of:
(a) determining the level of RhoGEF12 protein, the level of RhoGEF12 transcript and/or the level of activity of RhoGEF12 in a cell;
(b) contacting said cell or a cell of the same cell population with a test compound;
(c) determining the level of RhoGEF12 protein, the level of RhoGEF12 transcript and/or the level of activity of RhoGEF12 in said cell after contacting with the test compound; and
(d) comparing the level of RhoGEF12 protein, the level of RhoGEF12 transcript and/or the level of activity of RhoGEF12 determined in step (c) with the RhoGEF12 protein, the RhoGEF12 transcript and/or the RhoGEF12 activity level determined in step (a), wherein a decrease of the RhoGEF12 protein, the RhoGEF12 transcript and/or the RhoGEF12 activity level in step (c) as compared to step (a) indicates that the test compound is an inhibitor of RhoGEF12 or an inhibitor of an activator of RhoGEF12 suitable as a lead compound and/or as a medicament for the prevention and/or treatment of heart failure associated with cardiac hypertrophy and/or cardiac fibrosis and diseases associated therewith.

6. A method of identifying an inhibitor of Rho guanine nucleotide exchange factor 12 (RhoGEF12) or an inhibitor of an activator of RhoGEF12 suitable as a lead compound and/or as a medicament for the prevention and/or treatment of heart failure associated with cardiac hypertrophy and/or cardiac fibrosis and diseases associated therewith, comprising the steps of:
(a) determining the level of activity of an RhoGEF12 target molecule in a cell containing RhoGEF12;
(b) contacting said cell or a cell of the same cell population with a test compound;
(c) determining the level of activity of the RhoGEF12 target molecule in said cell after contacting with the test compound; and
(d) comparing the level of activity of the RhoGEF12 target molecule determined in step (c) with the level of activity of the RhoGEF12 target molecule determined in step (a), wherein a decreased activity of the target molecule in step (c) as compared to step (a) indicates that the test compound is an inhibitor of RhoGEF12 or an inhibitor of an activator of RhoGEF12 suitable as a lead compound and/or as a medicament for the prevention and/or treatment of heart failure associated with cardiac hypertrophy and/or cardiac fibrosis and diseases associated therewith.

7. The method of claim 6, wherein the RhoGEF12 target molecule is selected from the GTPase RhoA and the serum response factor (SRF).

8. The method of any one of claims 5 to 7, wherein said cell comprising the RhoGEF12 protein and/or transcript is a cardiomyocyte.

9. The method of any one of claims 5 to 8, further comprising optimising the pharmacological properties of an inhibitor of Rho guanine nucleotide exchange factor 12 (RhoGEF12) or an inhibitor of an activator of RhoGEF12 identified as lead compound.

10. The method of claim 9, wherein the optimisation comprises modifying the inhibitor of RhoGEF12 or the inhibitor of an activator of RhoGEF12 to achieve:
i) modified spectrum of activity, organ specificity, and/or
ii) improved potency, and/or
iii) decreased toxicity (improved therapeutic index), and/or
iv) decreased side effects, and/or
v) modified onset of therapeutic action, duration of effect, and/or
vi) modified pharmacokinetic parameters (resorption, distribution, metabolism and excretion), and/or
vii) modified physico-chemical parameters (solubility, hygroscopicity, color, taste, odor, stability, state), and/or
viii) improved general specificity, organ/tissue specificity, and/or
ix) optimised application form and route
by
(a) esterification of carboxyl groups, or
(b) esterification of hydroxyl groups with carboxylic acids, or
(c) esterification of hydroxyl groups to, e.g. phosphates, pyrophosphates or sulfates or hemi-succinates, or
(d) formation of pharmaceutically acceptable salts, or
(e) formation of pharmaceutically acceptable complexes, or
(f) synthesis of pharmacologically active polymers, or
(g) introduction of hydrophilic moieties, or
(h) introduction/exchange of substituents on aromates or side chains, change of substituent pattern, or
(i) modification by introduction of isosteric or bioisosteric moieties, or
(j) synthesis of homologous compounds, or
(k) introduction of branched side chains, or
(l) conversion of alkyl substituents to cyclic analogues, or
(m) derivatisation of hydroxyl groups to ketales, acetales, or
(n) N-acetylation to amides, phenylcarbamates, or
(o) synthesis of Mannich bases, imines, or
(p) transformation of ketones or aldehydes to Schiff's bases, oximes, acetales, ketales, enolesters, oxazolidines, thiazolidines
or combinations thereof.

11. The method of any one of claims 2 to 10, wherein the heart failure is associated with cardiac hypertrophy and/or cardiac fibrosis results from diseases and conditions selected from the group consisting of arterial hypertension, valve disease, aortic coarctation, cardiomyophaty and myocardial infarction.

12. The method of any one of claims 2 to 11, wherein the diseases associated with said heart failure are selected from the group consisting of dyspnea, exercise intolerance, pulmonary edema, peripheral edema, nocturia, ascites and hepatomegaly.

13. A method of preventing or reducing hypertrophy and/or fibrosis of cardiomyocytes comprising: contacting said cardiomyocytes with an effective amount of an inhibitor of the Rho guanine nucleotide exchange factor 12 (RhoGEF12) or an inhibitor of an activator of RhoGEF12.

14. (canceled)
